(19) United States
(12) Patent Application Publication
(10) Pub. No.: US 2008/0045838 A1
(43) Pub. Date: Feb. 21, 2008

(54) Ultrasonic transducer array, ultrasonic probe, ultrasonic endoscope and ultrasonic diagnostic apparatus

(75) Inventor: Hiroaki Hyuga, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Minato-ku (JP)

(21) Appl. No.: 11/812,724

(22) Filed: Jun. 21, 2007

(30) Foreign Application Priority Data
Jun. 28, 2006 (JP) 2006-178482

(51) Int. Cl. A61B 8/00

(52) U.S. Cl. 600/463; 367/117; 600/101; 600/104; 600/462

(57) ABSTRACT

In an ultrasonic transducer array in which plural kinds of ultrasonic transducers having different areas of ultrasonic transmission/reception surfaces are arranged, electric impedances among the ultrasonic transducers are made substantially equal. The ultrasonic transducer array, in which at least two kinds of ultrasonic transducers having different areas of ultrasonic transmission/reception surfaces from one another and having substantially equal total thicknesses to one another are arranged, includes: a first ultrasonic transducer; and a second ultrasonic transducer having a larger number of layers than that of the first ultrasonic transducer and a smaller area of an ultrasonic transmission/reception surface than that of the first ultrasonic transducer.

### BACKGROUND OF THE INVENTION

1. Field of the invention

The present invention relates to an ultrasonic transducer array for transmission and reception of ultrasonic waves, and an ultrasonic probe and an ultrasonic endoscope including the ultrasonic transducer array, and further, an ultrasonic diagnostic apparatus for generating ultrasonic images by using the ultrasonic probe or ultrasonic endoscope.

2. Description of a Related Art

Ultrasonic imaging technologies for generating images showing conditions within an object to be inspected by receiving ultrasonic echoes, that is, ultrasonic waves transmitted to the object and reflected by structures (organs and so on) within the object and performing signal processing thereon, are widely used in various fields including medical fields. An apparatus for ultrasonic imaging (called an ultrasonic diagnostic apparatus, ultrasonic imaging apparatus, or the like) is provided with a probe or ultrasonic endoscope for transmission and reception of ultrasonic waves. For imaging, the probe is used in contact with the object, or the ultrasonic endoscope is used by being inserted into the object.

In the ultrasonic probe and ultrasonic endoscope, vibrators (piezoelectric vibrators) having piezoelectric materials with electrodes formed on both sides thereof are generally used as ultrasonic transducers for transmission and reception of ultrasonic waves. When an electric field is applied to the electrodes of the vibrator, the piezoelectric material expands and contracts because of piezoelectric effect and generates ultrasonic waves. Accordingly, by driving plural vibrators while delaying the timing, an ultrasonic beam focused at a desired depth can be formed. Further, when the vibrator receives propagating ultrasonic waves, the vibrator expands and contracts to generate electric signals. The electric signals are used as reception signals of ultrasonic waves.

Recent years, an arrayed transducer (an ultrasonic transducer array), in which plural vibrators are arranged, has been used in the ultrasonic endoscope and ultrasonic endoscope. By using the ultrasonic transducer array, the transmission position or direction of an ultrasonic beam can be changed by controlling the amplitudes and amounts of delay of drive signals to be respectively applied to the plural vibrators without change in the position and orientation of the probe itself. Such scan system is called a phased array system or electronic scan system.

As a related technology, Japanese Patent Application Publication JP-A-7-203592 discloses an ultrasonic probe having, in order to improve the beam directivity in an ultrasonic probe having high resolving power, plural probe segments combined and arranged in a matrix form with ultrasonic transmission faces directed toward a predefined direction and displacing means for respectively displacing the ultrasonic radiation faces of the respective probe segments. That is, in JP-A-7-203592, the ultrasonic transmission faces of the probe segments (vibrators) are mechanically moved or tilted for easily focusing or deflecting the ultrasonic waves.

Further, recently, researches on a phased array, in which many vibrators are two-dimensionally arranged, have been increasingly made. This is because a focal point of an ultrasonic beam can be formed in a desired point within a three-dimensional space by transmitting plural ultrasonic waves from a two-dimensional region. Thereby, ultrasonic image information (volume data) on a three-dimensional space within the object can be acquired, and therefore, three-dimensional images can be constructed and image quality of ultrasonic images can be improved.

However, the size of matrix type two-dimensional phased array is larger than those of other arrays (one-dimensional array and so on). Further, with microfabrication and high density of elements, the fabrication of the two-dimensional phased array becomes difficult. Furthermore, the number of interconnections increases with increase in the number of elements, and therefore, a problem that a cable connected to the probe becomes thicker arises. It is especially difficult that such an array is applied to an ultrasonic endoscope. The ultrasonic endoscope is inserted into a living body, and there is a strong constriction on size.

On the other hand, researches on a so-called multirow array, in which plural one-dimensional arrays are arranged in parallel, have been also made. Although the number of arrays arranged in a multirow array is not so many as that in the matrix type two-dimensional phased array, ultrasonic beams focused with respect to two directions can be formed by using vibrators arranged in a two-dimensional region.

In the multirow array, the ultrasonic beam quality such as resolving power and the scanning volume remain inferior to those of the matrix type two-dimensional phased array. However, according to the multirow array, the number of elements and interconnections can be drastically reduced, and thereby, downsizing of the ultrasonic probe and ultrasonic endoscope can be realized and the costs can be reduced. Therefore, it is considered that there is a significant advantage in practical application of multirow array with a high performance.

Further, Japanese Patent Application Publication JP-P2000-139926A discloses an ultrasonic probe having ultrasonic transmitting and receiving means provided at a leading end of an insertion part to be inserted into a body cavity, for transmitting and receiving an ultrasonic beam, and a treatment tool lead-out opening from which a treatment tool such as a puncture needle can be led out toward a scan range of the ultrasonic beam by the ultrasonic transmitting and receiving means, and the ultrasonic probe includes ultrasonic deflecting means for deflecting the scan range of the ultrasonic beam by the ultrasonic transmitting and receiving means. That is, in JP-P2000-139926A, ultrasonic vibrators are arranged in three rows and ultrasonic waves with different phases are transmitted from the respective rows for deflection of the scan range of ultrasonic waves, and thereby, the ultrasonic beam is applied to the punctuation needle even when the punctuation needle is bent.

Furthermore, Japanese Patent Application Publication JP-P2004-57460A discloses an ultrasonic diagnostic apparatus having a continuous wave Doppler mode, and the ultrasonic diagnostic apparatus includes a vibrator array having plural vibrating elements arranged in an electronic scan direction and an elevation direction perpendicular to the electronic scan direction, and a transmission and reception control unit for controlling the operation of the plural vibrating elements. In the continuous wave Doppler mode, at least one group of transmission vibrating elements arranged in the electronic scan direction and at least one group of reception vibrating elements arranged in the electronic scan direction are set in different positions from each other in the elevation direction on the vibrator array. That is, in JP-P2004-57460A, the transmission aperture and the reception aperture are taken wider by alternate arrangement of transmission vibrating element row and reception vibrating element row.

Here, a configuration of a conventional multirow array will be explained with reference to FIGS. 20-23. FIG. 20(a) is a side view showing a conventional multirow array, FIG. 20(b) is a plan view thereof, and FIG. 21 is a partially enlarged plan view thereof. Further, FIGS. 22 and 23 show interconnection methods in the multirow array.

As shown in FIG. 20, the multirow array contains five element rows of L2-row, L1-row, C-row, R1-row, and R2-row in each of which 128 channels of ultrasonic transducers (also simply referred to as “elements”) are one-dimensionally arranged. Elements 901 are arranged in the C-row, elements 902 are arranged in the L1-row and the R1-row, and elements 903 are arranged in the L2-row and the R2-row. These elements are arranged on a backing layer 900. In the multirow array, the arrangement direction (X-axis direction) in the respective element rows is called an azimuth direction, and the direction perpendicular to the azimuth direction (Y-axis direction) is called an elevation direction.

As shown in FIGS. 22 and 23, each of the elements 901-903 includes a piezoelectric material layer 910 formed of lead (Pb) zirconate titanate (PZT) or the like, and electrodes 911 and 912 formed on the upper face and the lower face thereof. Generally, the electrodes 912 of all elements are connected to the ground potential. Further, the respective elements 901-903 are connected to interconnections 905-907 for supplying drive signals from the ultrasonic diagnostic apparatus main body to the elements and outputting reception signals generated by the elements to the ultrasonic diagnostic apparatus main body.

Regarding such a multirow array, in Wildes et al., “Elevation Performance of 1.25D and 1.5D Transducer arrays”, (IEEE TRANSACTIONS ON ULTRASONICS, FERROELECTRICS, AND FREQUENCY CONTROL, VOL. 44, NO. 5, SEPTEMBER 1997, pp. 1027-1037), the performance of multirow array is studied, in which the vibrator arrangement in the elevation direction and the interconnection method are changed. Here, according to the definition of Wildes et al., the dimensions of arrays can be explained as in the following (1) to (5).
(1) 1D array: Plural elements are arranged in one row (in the azimuth direction). Accordingly, the aperture diameter in the elevation direction (the element width in this case) is fixed, and the focal point of ultrasonic beam is formed by an acoustic lens or the like, and the focal length is fixed.
(2) 1.25D array: As shown in FIG. 22, plural elements are arranged in several rows (five rows in FIG. 22). The elements arranged in positions symmetric with respect to a central axis in the longitudinal direction of the array are commonly connected in parallel to the interconnections 905-907. Since the plural rows of elements are arranged in the elevation direction, the aperture diameter can be changed. Note that the focal point of ultrasonic beam is formed by an acoustic lens or the like, and the focal length is fixed.
(3) 1.5D array: Plural elements are arranged in several rows as is the case of the 1.25D array. The elements arranged in positions symmetric with respect to a center axis in the longitudinal direction of the array are commonly connected in parallel to the interconnections 905-907, and further, the focal point of ultrasonic beam is formed by electronic control. Accordingly, the focal point of ultrasonic beam can be dynamically changed. Note that the elements are symmetrically and commonly interconnected, and thereby, the ultrasonic beam cannot be deflected.
(4) 1.75D array: Plural elements are arranged in several rows, and further, the respective elements are independently interconnected. That is, as shown in FIG. 23, the plural elements arranged in the elevation direction are separately interconnected to the interconnections 904. Thereby, the ultrasonic beam can be deflected in the elevation direction in addition to dynamical change of the aperture diameter and the focal length of ultrasonic beam although the number of interconnections is larger than that of the 1.5D array.
(5) 2D array: Plural elements are arranged in substantially the same number in both the elevation direction and the azimuth direction to form a matrix. Accordingly, apodization, the focal length of ultrasonic beam in the three-dimensional space, and the transmission direction (deflection) of ultrasonic beam can be electrically controlled.

As to the 1.25D array, the 1.5D array, and the 1.75D array, since the degrees of freedom of control of ultrasonic beam are between those of the 1D array and 2D array, they are called the 1.25D array, the 1.5D array, and the 1.75D array.

In such a multirow array, in order to improve the quality of ultrasonic beam by reducing the grating lobes, the arrangement pitch of elements in the azimuth direction is typically set to be equal to or less than the wavelength of transmission ultrasonic waves.

On the other hand, with respect to the elevation direction, the widths W₁ to W₃ of the elements 901-903 are set to become narrower from inside (C-row) toward outside (L2-row and R2-row). Such arrangement is for improving the quality of ultrasonic beam, and arrangement methods called Fresnel arrangement, MIAE (Minimum Integrated Absolute time-delay Error) arrangement, and so on are known.

Refer to Wildes et al., which is incorporated herein by reference, for details of the Fresnel arrangement and MIAE arrangement.

In the multirow array shown in FIGS. 20 and 21, since the widths W₁ to W₃ of the elements 901-903 are different from one another while the lengths X₁ to X₃ of the elements 901-903 are common, the electric impedance values vary with respect to each row. Accordingly, degrees of electric impedance matching between the ultrasonic diagnostic apparatus main body and themselves are different with respect to each row, and thus, there are disadvantages that the transmission acoustic energy and reception sensitivity vary with respect to each row and the frequency characteristic as a system varies.

Further, Goldberg et al., “OPTIMIZATION OF SIGNAL-TO-NOISE RATIO FOR MULTILAYER PZT TRANSDUCERS”, (ULTRASONIC IMAGING 17, 1995, pp. 95-113) discloses an ultrasonic transducer having a multilayered structure (multilayer PZT transducer) in which the impedance of the ultrasonic transducer is matched to an output impedance of a transmission circuit or a reactance of a coaxial cable by changing the number of piezoelectric material layers. The ultrasonic transducer having a multilayered structure refers to an ultrasonic transducer having a structure in which plural piezoelectric material layers and plural electrode layers are alternately stacked as shown in FIG. 1 of Goldberg et al. In the ultrasonic transducer, electrodes are formed such that the sets of vibrator structures, each of which is configured by a piezoelectric material layer and two electrode layers, are connected electrically in parallel.

Here, in comparison between an N-layer ultrasonic transducer and a single-layer ultrasonic transducer having the equal sizes of the entire element (bottom area and thickness), the former has N-times the number of layers and 1/N of the thickness of each layer of the latter. Therefore, the electric impedance Z of the N-layer ultrasonic transducer is 1/N²-times the electric impedance Z_{T} of the single-layer ultrasonic transducer. Accordingly, in Goldberg et al., the number of layers of the ultrasonic transducer having the multilayered structure is optimized according to the following principle. That is, in an N_{TX}-layer ultrasonic transducer, the energy (acoustic output) of ultrasonic waves to be transmitted becomes the maximum when Z_{T}/N_{TX}²is equal to the output impedance of the transmission circuit. On the other hand, in an N_{RX}-layer ultrasonic transducer, the reception signal (voltage) becomes the maximum when Z_{T}/N_{RX}² is equal to the reactance of the coaxial cable. Therefore, when the number of layers of the ultrasonic transducer satisfies the geometric mean (N_{TX}-N_{RX})^{1/2}, the transmission and reception sensitivity of ultrasonic waves (pulse echo signals) becomes the maximum.

### SUMMARY OF THE INVENTION

Accordingly, in view of the above-mentioned points, a purpose of the present invention is, in an ultrasonic transducer array in which plural kinds of ultrasonic transducers having different areas of ultrasonic transmission/reception surfaces are arranged, to make electric impedances among the ultrasonic transducers substantially equal.

In order to achieve the above-mentioned purpose, an ultrasonic transducer array according to one aspect of the present invention is an ultrasonic transducer array in which at least two kinds of ultrasonic transducers having different areas of ultrasonic transmission/reception surfaces from one another and having substantially equal total thicknesses to one another are arranged, and the ultrasonic transducer array includes: a first ultrasonic transducer; and a second ultrasonic transducer having the larger number of layers than that of the first ultrasonic transducer and a smaller area of an ultrasonic transmission/reception surface than that of the first ultrasonic transducer.

In the present application, the expression that electric impedances are substantially equal includes not only the case where values in comparison are strictly equal but also the case where they are equal within a predetermine range of error, that is, those values are generally equal. For example, when variation between the values in comparison is within ±30%, it can be said that they are substantially equal.

According to the present invention, the numbers of layers of the ultrasonic transducers having the smaller areas are increased in the plural kinds of ultrasonic transducers having different areas of ultrasonic transmission/reception surfaces from one another and having substantially equal total thicknesses to one another, and thereby, the electric impedances among the ultrasonic transducers can be made substantially equal. Further, electric impedance matching between the respective ultrasonic transducers and the ultrasonic diagnostic apparatus main body are easily achieved, and thereby, the transmission acoustic energy can be increased in a high frequency. Therefore, by using an ultrasonic probe or ultrasonic endoscope including such an ultrasonic transducer array in an ultrasonic diagnostic apparatus, quality of an ultrasonic beam to be transmitted can be improved and good quality ultrasonic images can be easily generated based on the acquired reception signals.

### BRIEF DESCRIPTION OF THE DRAWINGS

FIG. 1 shows a configuration of an ultrasonic transducer array according to the first embodiment of the present invention;

FIG. 2 shows a configuration of an ultrasonic probe according to one embodiment of the present invention;

FIG. 3A shows a configuration example of an ultrasonic diagnostic apparatus according to one embodiment of the present invention;

FIG. 3B shows a configuration example of a reception signal processing unit as shown in FIG. 3A;

FIG. 4 is a partially sectional perspective view of an ultrasonic transducer having a multilayered structure;

FIG. 5 is a table showing simulation results of comparative example 1 and working examples 1 and 2;

FIG. 6 shows a configuration of an ultrasonic transducer array according to the second embodiment of the present invention;

FIG. 7 is a partially enlarged plan view of the ultrasonic transducer array shown in FIG. 6;

FIG. 8 shows a configuration of an ultrasonic transducer array according to the third embodiment of the present invention;

FIG. 9 is a table showing simulation results of comparative example 2 and working examples 3 and 4;

FIG. 10 shows a configuration of an ultrasonic transducer array according to the fourth embodiment of the present invention;

FIG. 11 is a partially enlarged plan view of the ultrasonic transducer array shown in FIG. 10;

FIG. 12 shows a configuration of an ultrasonic transducer array according to the fifth embodiment of the present invention;

FIG. 13 is a partially enlarged plan view of the ultrasonic transducer array shown in FIG. 12;

FIG. 14 is a schematic diagram showing a configuration of an ultrasonic endoscope according to one embodiment of the present invention;

FIG. 15 is an enlarged schematic diagram showing a leading end of an insertion part shown in FIG. 14;

FIG. 16 is a plan view showing an example of an ultrasonic transducer array in which plural kinds of elements are asymmetrically arranged;

FIG. 17 is a plan view showing an example of an ultrasonic transducer array in which plural kinds of oval elements are arranged;

FIG. 18 is a plan view showing an example of an ultrasonic transducer array in which plural kinds of polygonal elements are arranged;

FIG. 19 is a plan view showing an example of an ultrasonic transducer array in which plural kinds of annular elements are arranged;

FIG. 20 is a plan view showing a conventional ultrasonic transducer array of Fresnel arrangement;

FIG. 21 is a partially enlarged plan view of the ultrasonic transducer array shown in FIG. 20;

FIG. 22 is a side view showing arrangement and connection methods of elements in 1.25D array or 1.5D array; and

FIG. 23 is a side view showing arrangement and connection methods of elements in 1.75D array.

### DESCRIPTION OF THE PREFERRED EMBODIMENTS

Hereinafter, preferred embodiments of the present invention will be explained in detail with reference to the drawings. The same reference numerals will be assigned to the same component elements and the description thereof will be omitted.

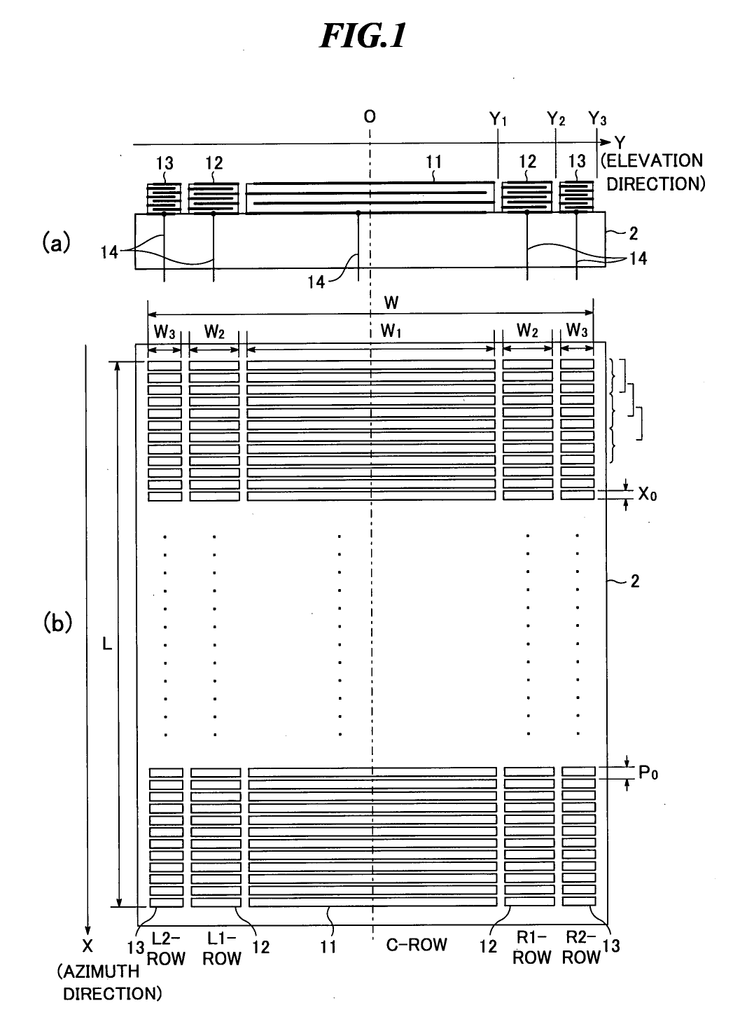

FIG. 1 schematically shows an ultrasonic transducer array according to the first embodiment of the present invention. FIG. 1(a) is a side view showing a state in which the ultrasonic transducer array according to the embodiment is provided on a backing layer, and FIG. 1(b) is a plan view thereof.

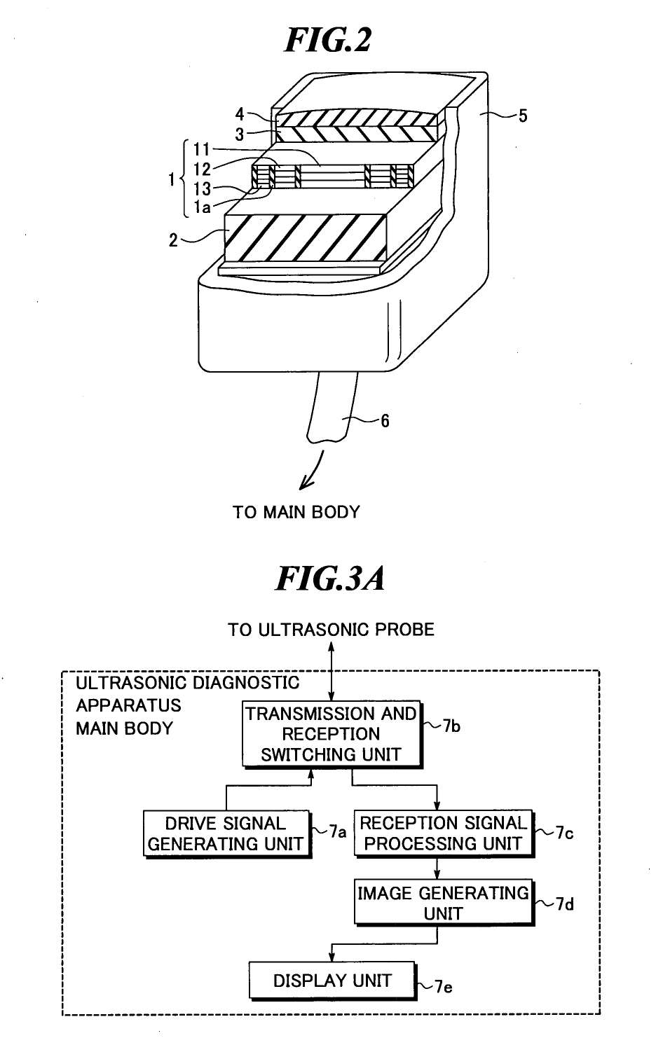

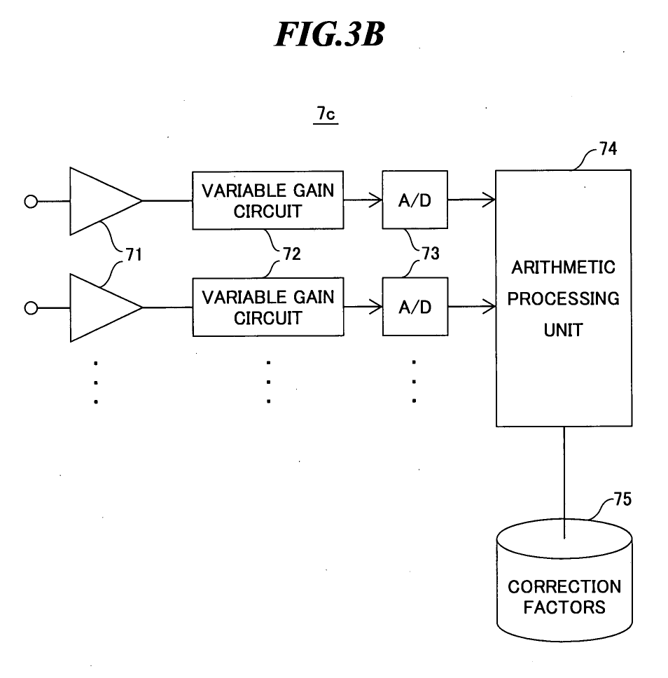

Further, FIG. 2 is a partially sectional perspective view showing an ultrasonic probe according to one embodiment of the present invention, and FIG. 3A is a block diagram showing an ultrasonic diagnostic apparatus according to one embodiment of the present invention. The ultrasonic transducer array shown in FIG. 1 is provided for use in the ultrasonic probe as shown in FIG. 2, an ultrasonic endoscope, which will be described later, and so on.

As shown in FIG. 2, the ultrasonic probe according to the embodiment includes an ultrasonic transducer array 1, a backing layer 2, and an acoustic matching layer 3. Further, the ultrasonic probe may include an acoustic lens 4 according to need. These parts are accommodated in a casing 5. Further, interconnections drawn from the ultrasonic transducer array 1 are connected to an electronic circuit contained in an ultrasonic imaging apparatus man body via a cable 6.

The ultrasonic transducer array 1 includes plural ultrasonic transducers 11-13 that expand and contract to generate ultrasonic waves when drive signals are supplied thereto, and receive ultrasonic waves propagating from an object to be inspected and output electric signals (reception signals). A filler la such as urethane resin or epoxy resin may be provided between and around the ultrasonic transducers 11-13 for protection of the respective ultrasonic transducers and suppression of unwanted propagation of ultrasonic waves (e.g., propagation of ultrasonic waves within the arrangement surface of the vibrators). As below, one ultrasonic transducer is also simply referred to as “element”. The structure of the elements 11-13 will be explained later in detail.

The backing layer 2 is formed of a material having large acoustic attenuation such as an epoxy resin containing ferrite powder, metal powder, or PZT powder, or rubber containing ferrite powder, and promotes attenuation of unwanted ultrasonic waves generated from the ultrasonic transducer array 1.

The acoustic matching layer 3 is formed of Pyrex (registered trademark) glass or an epoxy resin containing metal powder, which easily propagates ultrasonic waves, for example, and resolves the mismatch of acoustic impedances between the object as a living body and the ultrasonic transducer. Thereby, the ultrasonic waves transmitted from the ultrasonic transducers efficiently propagate within the object.

The acoustic lens 4 is formed of silicon rubber, for example, and focuses an ultrasonic beam, which is transmitted from the ultrasonic transducer array 1 and propagates the acoustic matching layer 3, at a predetermined depth within the object. In the ultrasonic transducer array according to the embodiment as explained below, the acoustic lens 4 is not necessarily provided because the ultrasonic beam can be focused by electronic control, however, the acoustic lens 4 may be simultaneously used for improvement in the focus effect of ultrasonic beam.

As shown in FIG. 3A, the ultrasonic diagnostic apparatus main body includes a drive signal generating unit 7a, a transmission and reception switching unit 7b, a reception signal processing unit 7c, an image generating unit 7d, and a display unit 7e. The drive signal generating unit 7a includes plural pulsers for generating drive signals to be respectively supplied to the elements provided in the ultrasonic transducer array 1 of the ultrasonic probe. Further, the transmission and reception switching unit 7b switches between the output of drive signals to the ultrasonic probe and the input of reception signals from the ultrasonic probe.

As shown in FIG. 3B, the reception signal processing unit 7c includes preamplifiers 71, variable gain circuits 72 such as amplifiers or attenuators for adjusting the signal levels according to need, A/D converters 73, an arithmetic processing unit 74, and so on, and performs predetermined signal processing such as pre-amplification, level adjustment, A/D conversion, phase adjustment and signal addition, and envelope detection on the reception signals outputted from the respective elements of the ultrasonic probe. Also, the arithmetic processing unit 74 may adjust the signal levels according to correction factors stored in a storage unit 75.

Referring to FIG. 3A again, the image generating unit 7d generates image data by performing scan format conversion or the like on the reception signals that have been subjected to the predetermined signal processing. The display unit 7e displays ultrasonic images based on the generated image data.

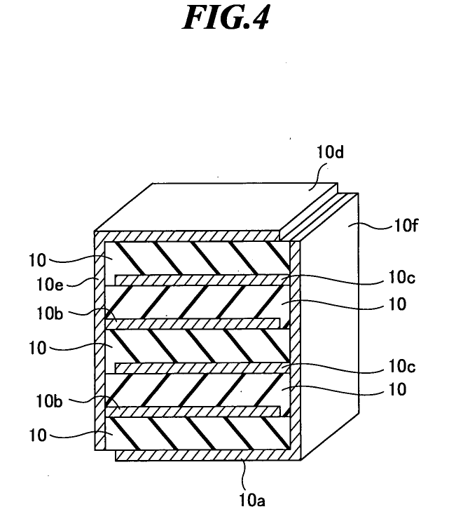

With reference to FIG. 1 again, the elements 11-13 arranged in the ultrasonic transducer array 1 have multilayered structures. Here, the structure of the multilayer ultrasonic transducer will be explained by referring to FIG. 4. FIG. 4 is a partially sectional perspective view showing a five-layer ultrasonic transducer.

The ultrasonic transducer shown in FIG. 4 has a lower electrode layer 10a, five piezoelectric material layers 10, internal electrode layers 10b and 10c alternately inserted between the piezoelectric material layers 10, an upper electrode layer 10d, and side electrodes 10e and 10f.

The piezoelectric material layer 10 is formed of a piezoelectric material such as lead (Pb) zirconate titanate (PZT). Further, the internal electrode layers 10b and the upper electrode layer 10d are formed to be connected to one side electrode 10e and insulated from the other side electrode 10f. Furthermore, the internal electrode layers 10c and the lower electrode layer 10a are formed to be connected to one side electrode 10f and insulated from the other side electrode 10e. Since the electrodes of the ultrasonic transducer are formed as described above, five sets of electrode for applying electric fields to the five piezoelectric material layers 10 are connected in parallel. In FIG. 4, insulating regions are formed by covering one end of the internal electrode 10b or 10c with the piezoelectric material layer 10 thereon. However, the ends thereof may be extended to the side surfaces of the piezoelectric material layer 10 and one end of the internal electrode 10b or 10c may be separately covered by an insulating film on the side surface.

In the ultrasonic transducer having N piezoelectric material layers 10 (N=5 in FIG. 4), the number of the piezoelectric material layers is N-times and the thickness of each piezoelectric material layer is 1/N compared to a single-layer ultrasonic transducer (N=1). Therefore, the electric impedance Z of the N-layer ultrasonic transducer is 1/N²-times the electric impedance Z_{T} of the single-layer ultrasonic transducer.

With reference to FIG. 1 again, the ultrasonic transducer array according to the embodiment includes three kinds of elements 11-13 arranged in five rows of L2-row, L1-row, C-row, R1-row, and R2-row. As below, the arrangement direction of elements in the respective rows (X-axis direction) is referred to as “azimuth direction” and the direction perpendicular thereto (Y-axis direction) is referred to as “elevation direction”.

In each row, 128 channels of ultrasonic transducers (elements) are arranged at an arrangement pitch P₀. The arrangement pitch P₀ is designed so as to be equal to or less than the half of the wavelength of transmission ultrasonic waves in consideration of generation angle of the grating lobes in the electronic sector scan system. For example, assuming that the sound speed in the living body is about 1500 m/s, when the frequency of the transmission ultrasonic waves is 5 MHz, the wavelength thereof is about 0.3 mm, and the half of the wavelength of the ultrasonic waves is 0.15 mm. In the embodiment, the arrangement pitch P₀ is 0.15 mm.

The widths W₁ to W₃ and positions Y₁ to Y₃ of the elements 11-13 (FIG. 1) are designed in Fresnel arrangement. The Fresnel arrangement is one of the arrangement methods, by which the widths W₁ to W₃ of the elements become narrower from the center toward outside in the elevation direction. Further, these elements 11-13 are designed such that the smaller the area of the ultrasonic transmission/reception surface becomes, the larger the number of layers becomes. The reason of changing the number layers in this manner is as follows. That is, when the Fresnel arrangement is adopted, the areas of the ultrasonic transmission/reception surfaces of the elements 11-13 change from one another, and thereby, the electric impedance values become different from one another. On this account, it becomes difficult to match the electric impedances between the elements and the ultrasonic diagnostic apparatus main body shown in FIG. 3A (specifically, the drive signal generating unit 7b, the reception signal processing unit 7c, and the cable 6). Accordingly, in the embodiment, in order to reduce the variation in electric impedance among elements, the number of layers is made larger as the element has the smaller area of the transmission/reception surface. Thereby, it becomes easier to match the respective electric impedances between the elements and the ultrasonic diagnostic apparatus main body, and the transmission and reception efficiency of ultrasonic waves can be improved.

Furthermore, as shown in FIG. 1(a), the elements 11-13 arranged in each row are independently connected to interconnections 14. Drive signals generated in the ultrasonic diagnostic apparatus main body are respectively supplied to the elements 11-13 via the interconnections 14.

When ultrasonic waves are transmitted by using such an ultrasonic transducer array, the elements 11-13 contained in one or some columns (e.g., three to five columns) arranged along the elevation direction are set as one set of drive elements to be simultaneously used, and the elements are driven while predetermined delay times are provided to the drive signals to be supplied to the elements. Thereby, an ultrasonic beam having a focal point at a desired depth can be transmitted in a desired direction. Further, the drive elements are sequentially driven while the set positions of the set of drive elements are moved in the azimuth direction, and thus, the object can be scanned with the ultrasonic beam by electronic control. In this regard, the present set of drive elements may be set such that the elements are not overlapped with the previous set of drive elements. For example, the elements of the first to third columns are used in the first transmission, the elements of the fourth to sixth columns are used in the second transmission, and the elements of the seventh to ninth columns are used in the third transmission. Alternatively, the present set of drive elements may be set such that the elements are partly overlapped with the previous set of drive elements. For example, the elements of the first to third columns are used in the first transmission, the elements of the third to fifth columns are used in the second transmission, and the elements of the fifth to seventh columns are used in the third transmission.

Next, specific design values of the ultrasonic transducer array according to the embodiment will be explained in comparison with design values in a conventional ultrasonic transducer array. Here, the design values as below are determined on the assumption that the ultrasonic transducer array is connected to an ultrasonic diagnostic apparatus under the following conditions.

<Pulser (drive signal generating unit)>
Output voltage: 100V
Output impedance: 30Ω

<Coaxial Cable>
Length: 3.4 m
Capacity: 100 pF/m
Impedance: 94Ω (5 MHz)

<Preamplifier (reception signal processing unit)>
Input impedance: 10 MΩ

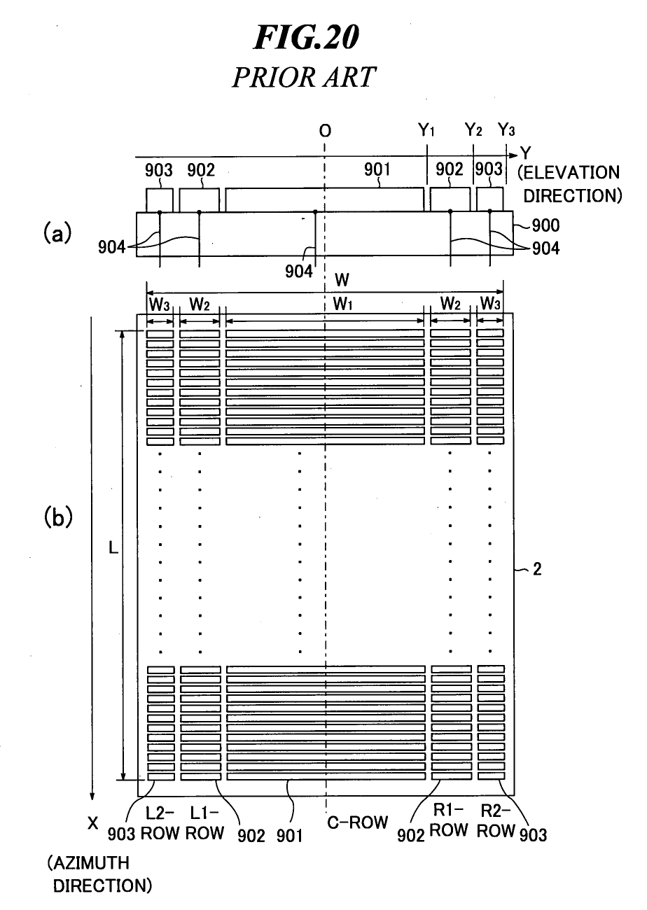

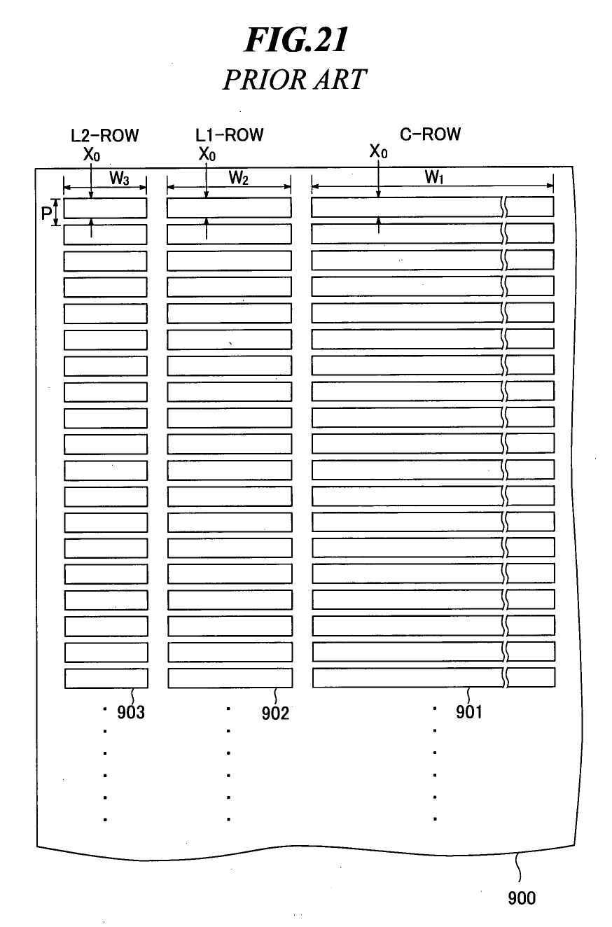

The sizes (lengths and widths) of the elements 11-13 contained in the ultrasonic transducer array shown in FIG. 1 are the same as those of the elements 901-903 shown in FIGS. 20 and 21, and the elements 11-13 are different from the elements shown in FIGS. 20 and 21 in that the elements 11-13 have multilayered structures.

The design values of the ultrasonic transducer array shown in FIG. 1 are as follows.
Arrangement Method: Fresnel arrangement
Interconnection Method: Independent interconnection
Planar Size of Elements:

<C-row (elements 11 and 901)>
Width W₁=2.88 mm
Length X₀=0.12 mm

<L1-row, R1-row (elements 12 and 902)>
Width W₂=0.57 mm
Length X₀=0.12 mm

<L2-row, R2-row (elements 13 and 903)>
Width W₃=0.43 mm
Length X₀=0.12 mm
Element Interval in Elevation Direction: 0.03 mm (30 μm)
Arrangement pitch in Azimuth Direction: P₀=0.15 mm
Element Interval in Azimuth Direction: 0.03 mm (30 μm)

Further, the relative dielectric constant εᵣ of the respective elements 901-903 is 3600. In FIG. 20, the thickness of the respective elements 901-903 is 0.36 mm. From the values and the sizes of the respective elements, at the resonant frequency of 5 MHz, the electric impedances Z₉₀₁ to Z₉₀₃ of the respective elements 901-903 are as follows.

<Element 901 (C-row)>
Z₉₀₁=326Ω

<Element 902 (L1-row, R1-row)>
Z₉₀₂=1647Ω

<Element 903 (L2-row, R2-row)>
Z₉₀₃=2183Ω

On the other hand, in the embodiment, the numbers of layers N₁₁ to N₁₃ of the respective elements 11-13 are determined such that the electric impedances Z₁₁ to Z₁₃ of the respective elements are as close as possible to about 53Ω as the geometric mean between the output impedance (30Ω) of the drive signal generating unit and the impedance (94Ω) of the cable.

As a result, the electric impedances of the respective elements 11-13 are as follows.

<Element 11 (C-row)>
N₁₁=3
Z₁₁=Z₉₀₁/3²−36.2Ω

<Element 12 (L1-row, R1-row)>
N₁₂=6
Z₁₂=Z₉₀₂/6²≈45.8Ω

<Element 13 (L2-row, R2-row)>
N₁₃=7
Z₁₃=Z₉₀₃/7²≈44.6Ω

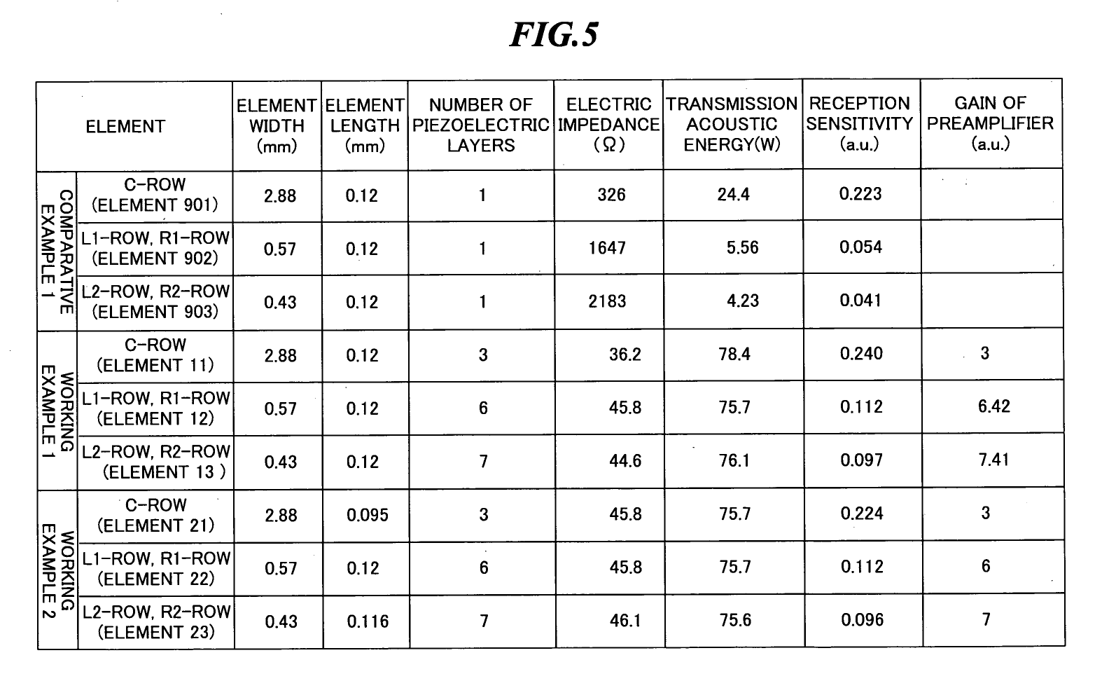

As working example 1, the characteristics of the ultrasonic transducer array according to the embodiment shown in FIG. 1 are simulated under the above-mentioned apparatus conditions. Further, as comparative example 1, the characteristics of the conventional ultrasonic transducer array shown in FIG. 20 are simulated under the same apparatus conditions. Thereby, the transmission and reception characteristics shown in FIG. 5 are obtained. Here, the reception sensitivity shows a relative reception voltage when sound waves having constant sound pressure are received in arbitrary unit (a.u.).

As clearly found from FIG. 5, in comparative example 1, the electric impedances largely vary among the elements 901-903, that is, 100×(Z₉₀₃-Z₉₀₁)/Z₉₀₃≈85.0% at the maximum. Further, the effect of transmission acoustic energy by the element 901 in the C-row is dominant. Even when an attempt to form an ultrasonic beam with such elements is made, the acoustic energy transmitted from the elements arranged outside (in the L1-row, R1-row, L2-row, R2-row) is not effectively acted thereon. That is, the entire aperture of the ultrasonic transducer array is not effectively utilized. Further, when the ultrasonic beam is deflected, the symmetry of the acoustic energy transmitted from the elements 901-903 is lost with respect to the transmission direction of the ultrasonic beam, and thus, the beam quality is deteriorated and the formation of good quality images becomes difficult.

On the other hand, in working example 1, the electric impedances among the elements 11-13 are substantially made equal, and variation is 100×(Z₁₂-Z₁₁)/Z₁₂≈20.1% at the maximum. Since the electric impedances of the elements 11-13 are made close to the output impedance of the transmission circuit, the magnitude of the transmission acoustic energy itself can be significantly improved. Further, the transmission acoustic energies of the elements 11-13 are nearly the same.

Meanwhile, regarding the reception sensitivity, although the reception sensitivity values themselves are improved compared to those in comparative example 1, slight variation remains with respect to each element. Here, when the reception sound pressure is constant, the reception voltage is proportional to the thickness of the piezoelectric material layer. Generally, in the case where the number of layers is increased to N-times, the reception voltages become 1/N. Accordingly, it is desirable that means for correcting the variation in reception voltage is provided.

For example, in the reception signal processing unit 7c as shown in FIG. 3B, preamplifiers 71 having gains (amplification factors), which are set for respective element rows, may be provided. The gain ratio among element rows may be set generally in correspondence with the ratio among numbers of layers, and may be further fine adjusted. For example, given that the gain corresponding to the element 11 in the C-row is G₁, the gain corresponding to the element 12 in the L1-row and R1-row is G₂, and the gain corresponding to the L2-row and R2-row is G₃, and these gains are set to satisfy G₁:G₂:G₃=3:6.42:7.41. Thereby, the levels of reception signals can be corrected to be nearly constant in all element rows.

Alternatively, as correcting means, variable gain circuits 72 such as amplifiers or attenuators may be provided at the downstream of the preamplifiers 71 in the reception signal processing unit 7c so as to set gains with respect to respective element rows. Or, the reception signals may be digitalized by A/D converters 73 provided at the downstream of the preamplifiers 71 or variable gain circuits 72, and arithmetic processing on the digital reception signals may be performed by an arithmetic processing unit 74 to correct the reception signals according to correction factors which correspond to the above-mentioned gain ratio. The correction factors have been previously stored in a storage unit 75.

Furthermore, such correction may be performed at the ultrasonic probe (or ultrasonic endoscope) side. That is, the preamplifiers or variable gain circuits for the respective elements, or the A/D converters and arithmetic processing unit may be provided in the ultrasonic probe (or ultrasonic endoscope) in which the ultrasonic transducer array is provided, and the gains or correction factors may be set to become the above-mentioned ratio.

As explained above, according to the embodiment, since the numbers of layers of the elements 11-13 are determined according to the areas of ultrasonic transmission/reception surfaces, the electronic impedances among the elements can be made substantially equal to one another. Thereby, it becomes easier to match between the electric impedances of the respective elements and the electric impedance of the apparatus side, and the transmission acoustic energy and reception sensitivity can be improved. Furthermore, regarding the reception signals outputted from the respective elements, variation in reception sensitivity can be reduced by adjusting gains with respect to the respective elements (respective element rows).

Consequently, all elements within the aperture can effectively act, and thus, ultrasonic beams with focal point that have been sufficiently narrowed can be transmitted and received. Further, even when the ultrasonic beam is deflected in the elevation direction, the ultrasonic beam with relatively good symmetry of acoustic energy with respect to the transmission direction, i.e., good quality ultrasonic beam can be formed. Therefore, good quality ultrasonic images can be formed based on ultrasonic image information obtained by such ultrasonic beams. Especially, ultrasonic image information (volume data) on the respective positions in the three-dimensional space within the object can be acquired by deflecting the ultrasonic beam without change in the position and orientation of the ultrasonic probe, and thus, three-dimensional images can be constructed at a high speed.

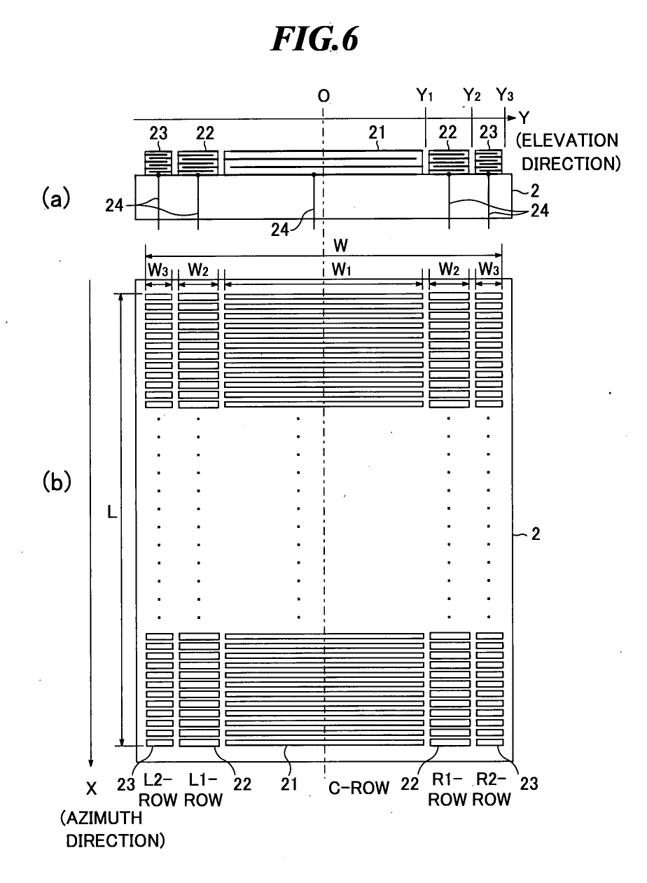

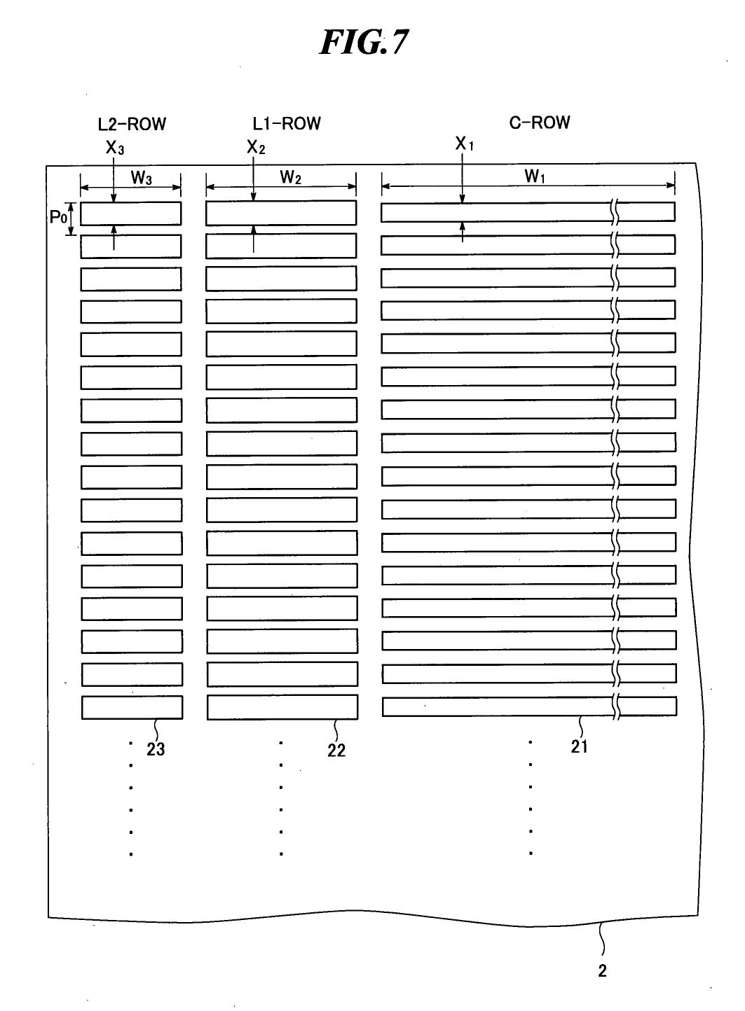

Next, an ultrasonic transducer array according to the second embodiment of the present invention will be explained with reference to FIGS. 6 and 7. FIG. 6(a) is a side view showing a state in which the ultrasonic transducer array according to the embodiment is provided on a backing layer, FIG. 6(b) is a plan view thereof, and FIG. 7 is a partially enlarged plan view showing ultrasonic transducers shown in FIG. 6(b).

As shown in FIG. 6, the ultrasonic transducer array according to the embodiment includes three kinds of elements 21-23 arranged in five rows of L2-row, L1-row, C-row, R1-row, and R2-row. These elements 21-23 are respectively connected to interconnections 24. The arrangement method (Fresnel arrangement), the interconnection method (independent interconnection), and the arrangement pitch of elements are the same as those shown in FIG. 1.

In the ultrasonic transducer array according to the embodiment, the lengths (the sizes in the azimuth direction) of the respective elements are changed from those of the ultrasonic transducer array shown in FIG. 1. In the first embodiment, although it is attempted to make the electric impedance among elements equal by adjusting the numbers of layers of the respective elements, it has limitations because the numbers of layers are integral numbers. Accordingly, in the embodiment, the lengths X₁ to X₃ of the elements are adjusted to satisfy S₂₁:S₂₂:S₂₃=1/N₂₁²:1/N₂₂²:1/N₂₃², where S₂₁ to S₂₃ represent the areas (length×width) of ultrasonic transmission/reception surfaces of the elements 21-23, respectively, and N₂₁ to N₂₃ represent the numbers of layers of them, respectively. Thereby, variation in the electric impedances Z₂₁ to Z₂₃ can be further reduced while maintaining the Fresnel arrangement (element sizes and element intervals in the elevation direction).

Specific design values of the ultrasonic transducer array according to the embodiment are as follows.

<Element 21 (C-row)>
N₂₁=3
Width W₁=2.88 mm
Length X₁=0.095 mm
Z₂₁=45.8Ω² (at a resonant frequency of 5 MHz)

### The value of electric impedance Z₂₁ is equivalent to about X₀/X₁ times the electric impedance Z₁₁ of the element 11 shown in FIG. 1.

<Element 22 (L1-row and R1-row)>
N₂₂=6
Width W₂=0.57 mm
Length X₂=0.120 mm
Z₂₂=45.8Ω (at a resonant frequency of 5 MHz)

### The value of electric impedance Z₂₂ is equivalent to about X₀/X₂ times the electric impedance Z₁₂ of the element 12 shown in FIG. 1.

<Element 23 (L2-row and R2-row)>
N₂₃=7
Width W₃=0.43 mm
Length X₃=0.116 mm
Z₂₃=46.1Ω (at a resonant frequency of 5 MHz)

### The value of electric impedance Z₂₃ is equivalent to about X₀/X₃ times the electric impedance Z₁₃ of the element 13 shown in FIG. 1.

As working example 2, the characteristics of the above-mentioned ultrasonic transducer array are simulated under the same apparatus conditions as those of working example 1 and comparative example 1. Thereby, results shown in FIG. 5 are obtained.

As shown in FIG. 5, the electric impedances among the elements 21-23 are substantially made equal (variation is 100× (Z₂₃-Z₂₁)/Z₂₃≈0.7% at the maximum) by adjusting the lengths of the elements in addition to the numbers of layers of the respective elements. Further, variation in transmission acoustic energy can be further reduced. Regarding the elements 21 and 23, the transmission acoustic energies are slightly lower because the lengths of elements are made smaller than those of the elements 11 and 13 in working example 1. However, matching between the electric impedances of the respective elements and the output impedance of the transmission circuit is good, and thus, the values significantly higher than those of comparative example 1 can be still maintained.

On the other hand, slight variation remains with respect to each element. Accordingly, in the embodiment, it is desirable that means for correcting variation in reception sensitivity is provided at the apparatus side or ultrasonic probe side as is the case of the first embodiment. The gain ratio among element rows can be generally regarded as the ratio among numbers of layers. For example, the reception sensitivity can be corrected to be nearly constant in all element rows by setting the gains G₁, G₂, G₃ corresponding to the C-row, the L1-row and R1-row, and the L2-row and R2-row to 3:6:7.

As explained above, according to the embodiment, since the numbers of layers and lengths of the elements are adjusted such that the electronic impedances among the elements are made substantially equal, variation in transmission acoustic energy and reception sensitivity can be further reduced. Further, in this regard, the electric impedances of the respective elements are made equal to the electric impedance of the apparatus side, and thus, the transmission acoustic energy and reception sensitivity can be further improved.

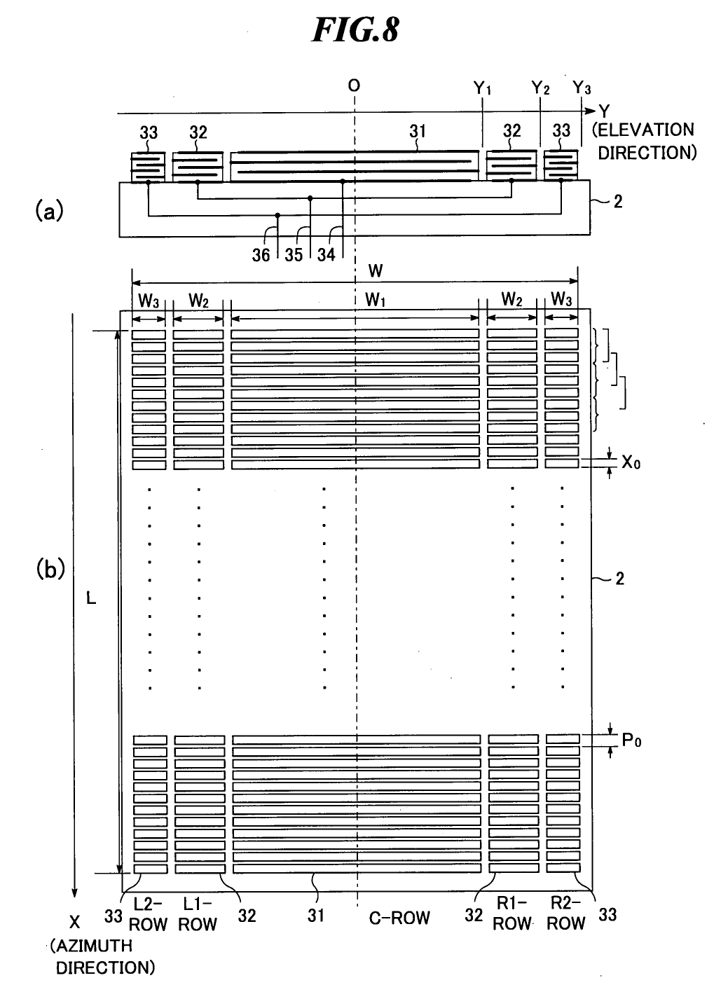

Next, an ultrasonic transducer array according to the third embodiment of the present invention will be explained with reference to FIG. 8. FIG. 8(a) is a side view showing a state in which the ultrasonic transducer array according to the embodiment is provided on a backing layer and FIG. 8(b) is a plan view thereof.

In the ultrasonic transducer array according to the embodiment, the interconnection method and the numbers of layers of the respective elements are changed from those of the ultrasonic transducer array shown in FIG. 1. The planar sizes of the respective elements and the arrangement method (Fresnel arrangement) are the same as those shown in FIG. 1.

As shown in FIG. 8(b), the ultrasonic transducer array includes three kinds of elements 31-33 arranged in five rows of L2-row, L1-row, C-row, R1-row, and R2-row. Further, as shown in FIG. 8(a), the elements 31 in the C-row are connected to an interconnection 34, the elements 32 in the L1-row and R1-row are connected to an interconnection 35, and the elements 33 in the L2-row and R2-row are connected to an interconnection 36. Thus, the elements arranged symmetrically with respect to the center in the elevation direction are commonly interconnected, and thereby, drive signals generated in the ultrasonic diagnostic apparatus main body are supplied to those elements with the same timing.

Specific design values of the ultrasonic transducer array according to the embodiment will be explained in comparison with design values in the conventional ultrasonic transducer array. The design values as below are determined on the assumption that the ultrasonic transducer array is connected to the ultrasonic diagnostic apparatus under the same conditions as those in the first embodiment.

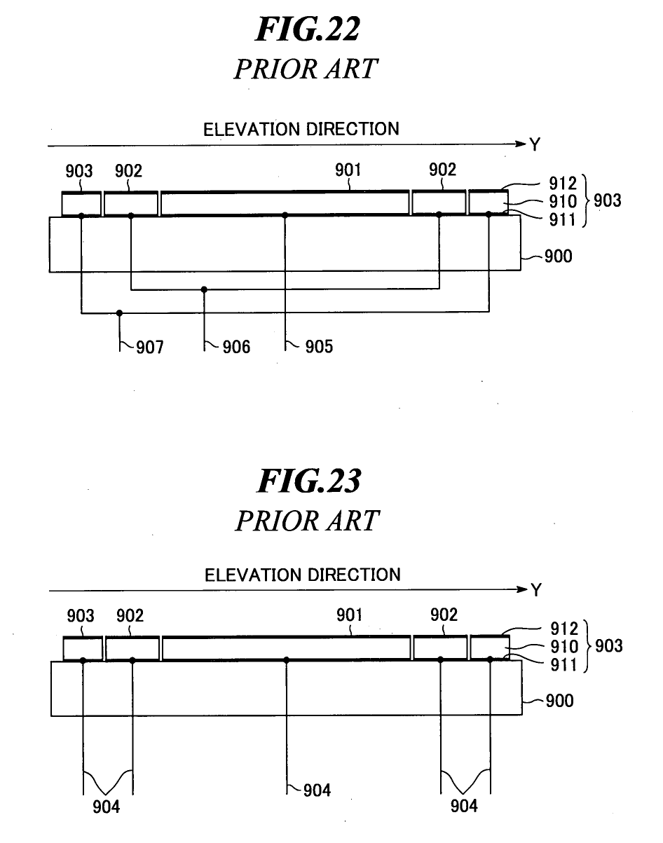

The design values of the common connection type ultrasonic transducer array shown in FIGS. 8 and 22 are as follows.
Arrangement Method: Fresnel arrangement
Interconnection Method: Common interconnection
Planar Size of Elements:

<Elements 31 connected to interconnection 34 (C-row)>

<Elements 901 connected to interconnection 905 (C-row)>
Width W₁=2.88 mm
Length X₀=0.12 mm

<Two elements 32 connected to interconnection 35 (L1-row, R1-row)>

<Two elements 902 connected to interconnection 906 (L1-row, R1-row)>
Width W₂=0.57×2 mm
Length X₀=0.12 mm

<Two elements 33 connected to interconnection 36 (L2-row, R2-row)>

<Two elements 903 connected to interconnection 907 (L2-row, R2-row)>
Width W₃=0.43×2 mm
Length X₀=0.12 mm
Element Interval in Elevation Direction: 0.039 mm (39 μm)
Arrangement pitch in Azimuth Direction: P₀=0.15 mm
Element Interval in Azimuth Direction: 0.03 mm (30 μm)

Further, the relative dielectric constant εᵣ of the respective elements 901-903 is 3600. In FIG. 22, the thickness of the respective elements 901-903 is 0.36 mm. From the values and the sizes of the respective elements, at the resonant frequency of 5 MHz, the synthesized electric impedances Z₉₀₁′ to Z₉₀₃′ of the elements connected to common interconnections are as follows.

<Element 901>
Z₉₀₁′=326Ω

<Two elements 902>
Z₉₀₂′=824Ω

<Two elements 903>
Z₉₀₃′=1092Ω

On the other hand, in the embodiment, the numbers of layers N₃₁ to N₃₃ of the respective elements 31-33 are determined such that the synthesized electric impedances Z₁₁ to Z₁₃ of the elements connected to common interconnections are as close as possible to about 53Ω as the geometric mean between the output impedance (30Ω) of the drive signal generating unit and the impedance (94Ω) of the cable.

As a result, the synthesized electric impedances of the respective elements connected to the respective interconnections are as follows.

<Elements 31 connected to interconnection 34 (C-row)>
N₃₁=3
Z₃₁=Z₉₀₁′/3²≈36.2Ω

<Two elements 32 connected to interconnection 35 (L1-row, R1-row)>
N₃₂=4
Z₃₂=Z₉₀₂′/4²≈51.5Ω

<Two elements 33 connected to interconnection 36 (L2-row, R2-row)>
N₃₃=5
Z₃₃=Z₉₀₃′/5²≈43.7Ω

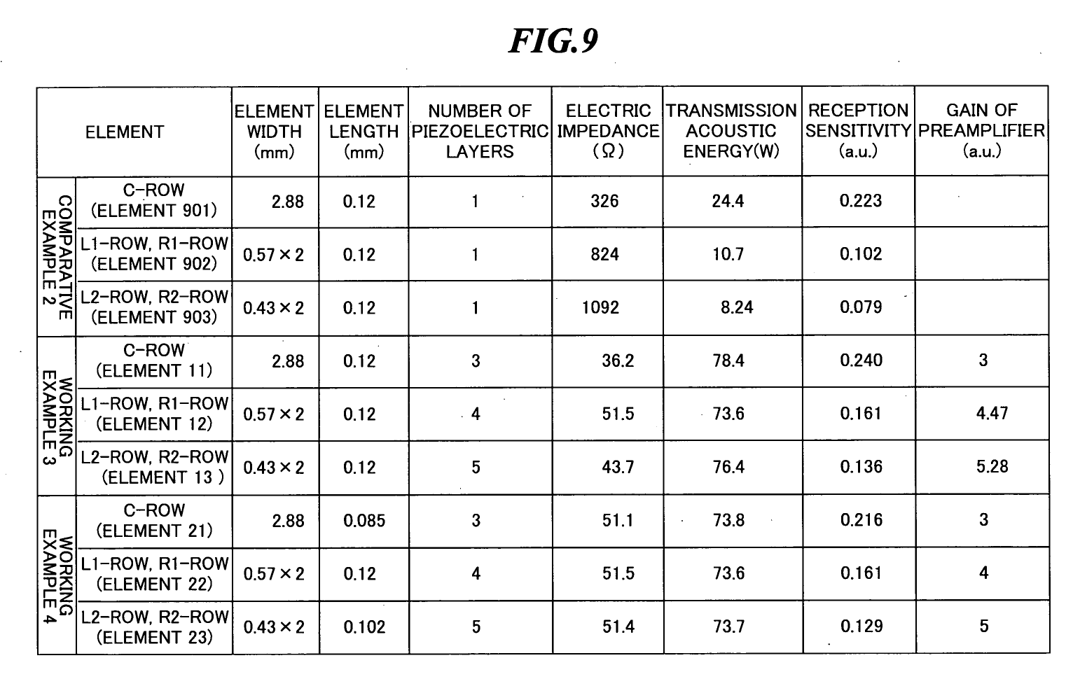

As working example 3, the characteristics of the ultrasonic transducer array according to the embodiment shown in FIG. 8 are simulated under the above-mentioned apparatus conditions. Further, as comparative example 2, the characteristics of the conventional ultrasonic transducer array shown in FIGS. 20 and 22 are simulated under the same apparatus conditions. Thereby, the transmission and reception characteristics shown in FIG. 9 are obtained.

As clearly found from FIG. 9, in comparative example 2, the synthesized electric impedances with respect to each of the interconnections 905-907 largely vary (100×(Z₉₀₃′-Z₉₀₁′)/Z₉₀₃′≈70.1% at the maximum). Further, the effect of transmission acoustic energy by the element 901 in the C-row is dominant, and the elements 902 in the L1-row and R1-row and the elements 903 in the L2-row and R2-row do effectively function. That is, the entire aperture of the ultrasonic transducer array is not effectively utilized. On this account, it may be impossible to form the focal point at a desired depth by dynamic focusing and the resolving power is reduced.

On the other hand, in working example 3, the synthesized electric impedances with respect to each of the interconnections 35-37 (i.e., the sums of capacitances) can be substantially made equal to one another (100×(Z₃₂-Z₃₁)/Z₃₂≈29.7% at the maximum) by adjusting the numbers of layers of the respective elements. Since the electric impedances with respect to each interconnection are made close to the output impedance of the transmission circuit, the magnitude of the transmission acoustic energy itself can be significantly improved. Further, the transmission acoustic energies among the elements 31-33 are nearly the same.

Meanwhile, regarding the reception sensitivity, although the reception sensitivity values themselves are improved compared to those in comparative example 2, slight variation remains with respect to each element. Accordingly, in the embodiment, it is desirable that means for correcting the variation in reception sensitivity is provided at the apparatus side or the ultrasonic probe side as is the case of the first embodiment. The gain ratio among element rows may be generally the ratio among numbers of layers, and may be further fine adjusted. For example, the reception sensitivity can be corrected to be nearly constant in all element rows by setting the gains G₁, G₂, G₃ corresponding to the C-row, the L1-row and R1-row, and the L2-row and R2-row to 3:4.47:5.28.

As explained above, according to the embodiment, variation in transmission acoustic energy and reception sensitivity can be reduced and the values can be improved. Accordingly, the entire aperture can effectively act and accurate dynamic focusing can be performed. Therefore, good quality ultrasonic images with high resolving power can be generated. Further, according to the embodiment, since the elements arranged symmetrically with respect to the center row are connected to common interconnections, the number of interconnections can be reduced, and the downsizing of the ultrasonic probe, the thinning down of the cable, and the reduction in costs can be realized.

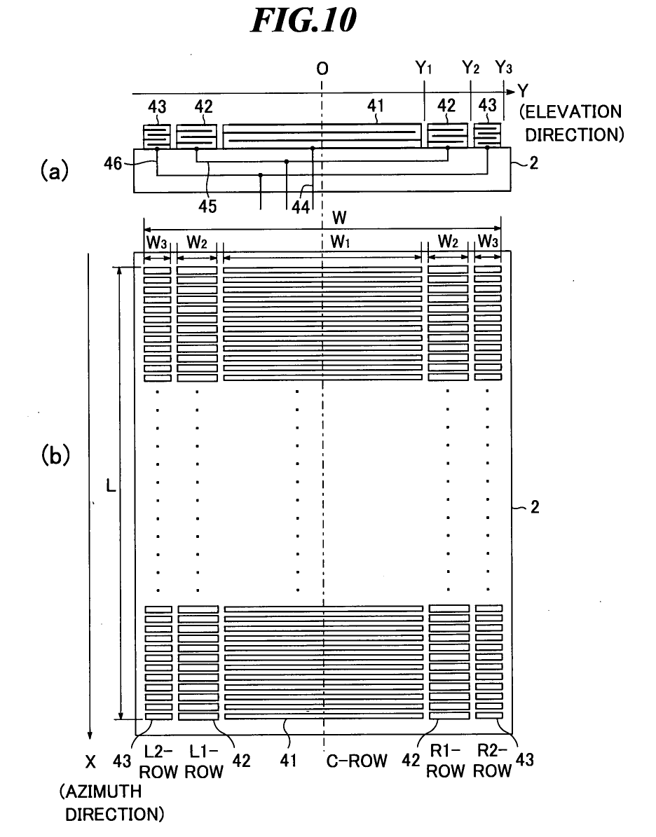

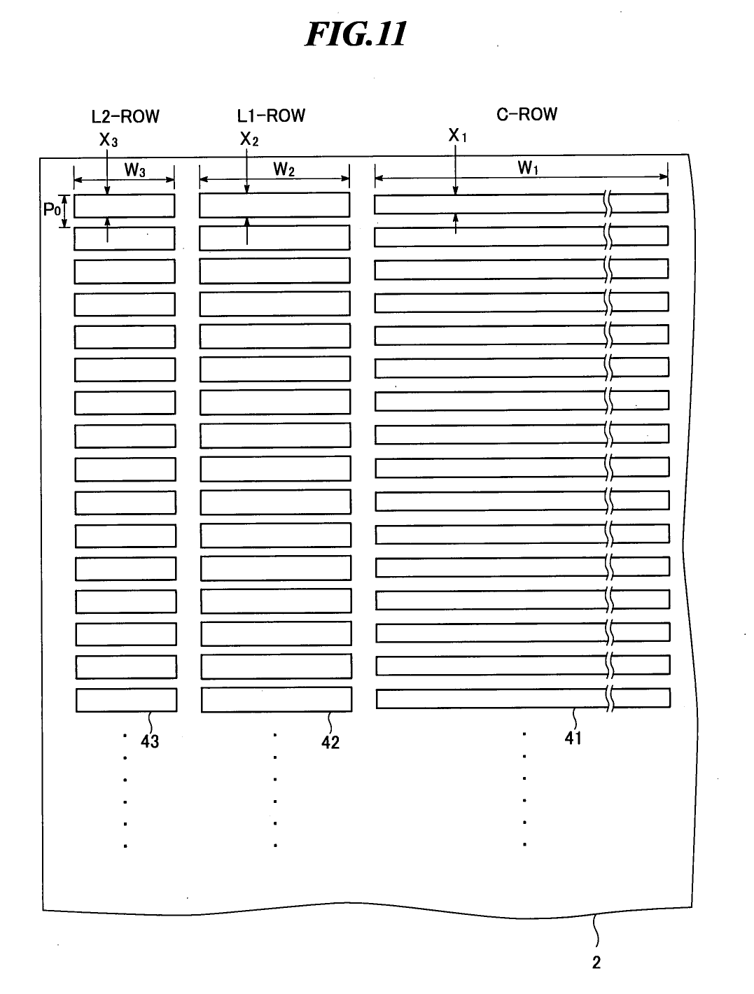

Next, an ultrasonic transducer array according to the fourth embodiment of the present invention will be explained with reference to FIGS. 10 and 11. FIG. 10(a) is a side view showing a state in which the ultrasonic transducer array according to the embodiment is provided on a backing layer, FIG. 10(b) is a plan view thereof, and FIG. 10 is a partially enlarged plan view showing ultrasonic transducers shown in FIG. 10(b).

As shown in FIG. 10(a), the ultrasonic transducer array according to the embodiment includes three kinds of elements 41-43 arranged in five rows of L2-row, L1-row, C-row, R1-row, and R2-row. Further, the elements 41 in the C-row are connected to an interconnection 44, the elements 42 in the L1-row and R1-row are connected to an interconnection 45, and the elements 43 in the L2-row and R2-row are connected to an interconnection 46. The arrangement method (Fresnel arrangement), the interconnection method (common interconnection), and the arrangement pitch of elements in the embodiment are the same as those shown in FIG. 8.

In the ultrasonic transducer array according to the embodiment, the lengths (the sizes in the azimuth direction) of the respective elements are changed from those of the ultrasonic transducer array shown in FIG. 8. That is, in the embodiment, the lengths X₁ to X₃ of the elements are adjusted to satisfy S₄₁:S₄₂:S₄₃=1/(N₄₁²·M₄₁):1/(N₄₂²·M₄₂):1/(N₄₃²·M₄₃), where S₄₁ to S₄₃ represent the areas (length×width) of ultrasonic transmission/reception surfaces of the elements 41-43, respectively, N₄₁ to N₄₃ represent the numbers of layers of them, respectively, and M₄₁ to M₄₃ represent the numbers of elements connected to the same interconnections. Thereby, variation in the synthesized electric impedances Z₄₁ to Z₄₃ with respect to each interconnection can be further reduced while maintaining the Fresnel arrangement (element sizes and element intervals in the elevation direction).

Specific design values of the ultrasonic transducer array according to the embodiment are as follows.

<Elements 41 connected to interconnection 44 (C-row)>
N₄₁=3
Width W₁=2.88 mm
Length X₁′=0.085 mm
Z₄₁=51.1Ω (at a resonant frequency of 5 MHz)

### The value of electric impedance Z₄₁ is equivalent to about X₀/X₁′ times the electric impedance Z₃₁ of the element 31 shown in FIG. 8.

<Two elements 42 connected to interconnection 45 (L1-row, R1-row)>
N₄₂=4
Width W₂=0.57 mm
Length X₂′=0.120 mm
Z₄₂=51.5Ω (at a resonant frequency of 5 MHz)

### The value of electric impedance Z₄₂ is equivalent to about X₀/X₂′ times the electric impedance Z₃₂ of the element 32 connected to the interconnection 35 shown in FIG. 8.

<Two elements 43 connected to interconnection 46 (L2-row, R2-row)>
N₄₃=5
Width W₃=0.43 mm
Length X₃′=0.102 mm
Z₄₃=51.4Ω (at a resonant frequency of 5 MHz)

### The value of electric impedance Z₄₃ is equivalent to about X₀/X₃′ times the electric impedance Z₃₃ of the element 33 connected to the interconnection 36 shown in FIG. 8.

As working example 4, the characteristics of the above-mentioned ultrasonic transducer array are simulated under the same apparatus conditions as those of working example 3 and comparative example 2. Thereby, results shown in FIG. 9 are obtained.

As shown in FIG. 9, the synthesized electric impedances with respect to each of the interconnections 44-46 (i.e., the sums of capacitances) are substantially made equal to one another (variation is 100×(Z₄₁-Z₄₂)/Z₄₂≈0.8% at the maximum) by adjusting the lengths of the elements in addition to the numbers of layers of the respective elements. Further, variation in transmission acoustic energy among the elements 41-43 can be further reduced. Regarding the elements 41 and 43, the transmission acoustic energies are slightly lower because the lengths of elements are made smaller than those of the elements 31 and 33 in working example 3. However, matching between the electric impedances of the respective sets of elements and the output impedance of the transmission circuit is good, and thus, the values significantly higher than those of comparative example 2 can be still maintained.

On the other hand, slight variation remains with respect to each element. Accordingly, in the embodiment, it is desirable that means for correcting variation in reception sensitivity is provided at the apparatus side or ultrasonic probe side as is the case of the first embodiment. The gain ratio among element rows can be generally regarded as the ratio among numbers of layers. For example, the reception sensitivity can be corrected to be nearly constant in all element rows by setting the gains G₁, G₂, G₃ corresponding to the C-row, the L1-row and R1-row, and the L2-row and R2-row to 3:4:5.

As explained above, according to the embodiment, since the numbers of layers and lengths of the elements are adjusted such that the synthesized electronic impedances with respect to each interconnection are made substantially equal, variation in transmission acoustic energy and reception sensitivity can be further reduced. Further, the synthesized electric impedances of the elements of the respective interconnections are made equal to the electric impedance of the apparatus side, and thus, the transmission acoustic energy and reception sensitivity can be further improved.

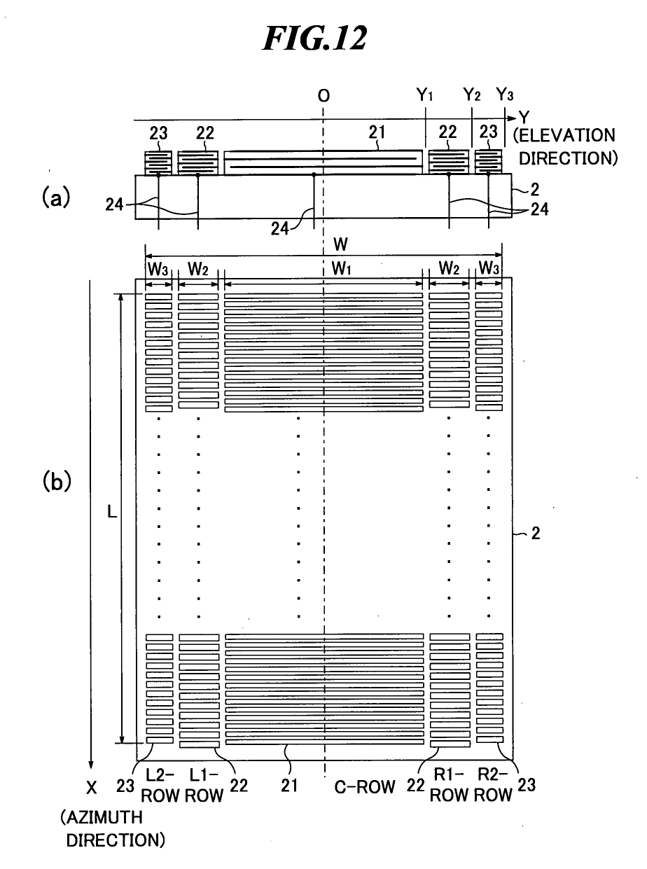

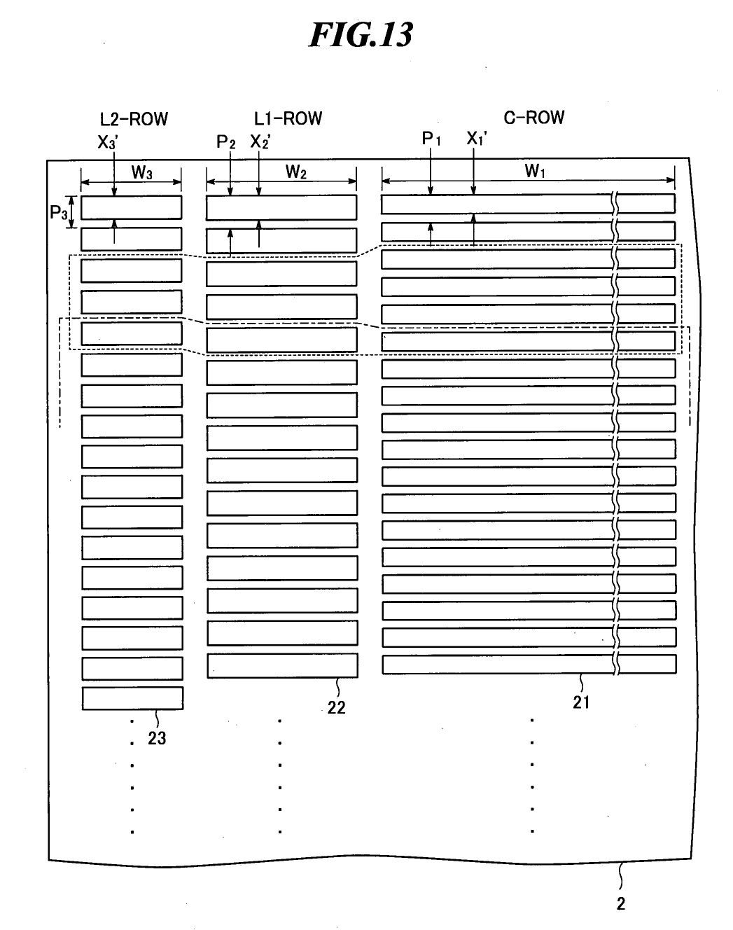

Next, an ultrasonic transducer array according to the fifth embodiment of the present invention will be explained with reference to FIGS. 12 and 13.

In the ultrasonic transducer array according to the embodiment, the arrangement of elements is changed from that of the ultrasonic transducer array shown in FIG. 6. That is, the element intervals in the azimuth direction are unified to 0.03 mm in the C-row where the elements 21 are arranged, the L1-row and the R1-row where the elements 22 are arranged, and the L2-row and the R2-row where the elements 23 are arranged. The element interval of 0.03 mm is designed such that the arrangement pitches P₁ to P₃ of the elements containing the lengths X₁ to X₃ of the elements 21-23 are equal to or less than the half of the wavelength of the transmission ultrasonic waves (e.g., 150 μm or less) at the maximum.

The sizes and the numbers of layers of the respective elements 21-23, the arrangement in the elevation direction (Fresnel arrangement), and the interconnection method (independent interconnection) are the same as those in the second embodiment.

Specifically, as shown in FIG. 13, 152 channels of elements 21 are arranged at the arrangement pitch P₁=0.125 μm (element length 0.095 μm+element interval 0.03 mm) in the central C-row. Further, 128 channels of elements 22 are arranged at the arrangement pitch P₂=0.15 mm (element length 0.12 m+element interval 0.03 mm) in the L1-row and R1-row. Furthermore, 132 channels of elements 23 are arranged at the arrangement pitch P₃=0.146 mm (element length 0.116 mm+element interval 0.03 mm) in the L2-row and R2-row.

When ultrasonic waves are transmitted using such an ultrasonic transducer array, the elements 21-23 contained in a predetermined range are set as one set of drive elements to be simultaneously used, and the elements are driven while predetermined delay times are provided to the elements. Thereby, an ultrasonic beam having a focal point at a desired depth can be transmitted in a desired direction. For example, as shown by the dotted line in FIG. 13, at the Nth transmission, the elements 23 contained in the third to fifth columns of the L2-row and R2-row, the elements 22 contained in the third to fifth columns of the L1-row and R1-row, and the elements 21 contained in the third to sixth columns of the C-row are set as drive elements. Further, for the next transmission, the present set of drive elements may be set such that the elements are not overlapped with the previous set of drive elements, or the present set of drive elements may be set such that the elements are partly overlapped with the previous set of drive elements as shown by the dashed-dotted line in FIG. 13.

As described above, according to the embodiment, since the elements are arranged at the same element intervals in the respective rows of C-row, L2-row, and R2-row, the filling ratio of elements to the entire area of the ultrasonic transducer array can be improved. Thereby, the transmission and reception sensitivity of ultrasonic waves and resolving power can be improved, and therefore, image quality of ultrasonic images generated in the ultrasonic diagnostic apparatus main body can be improved.

Note that the ultrasonic transducer array explained in the fourth embodiment of the present invention may be modified such that the intervals of the elements 41-43 are the same in the respective rows.

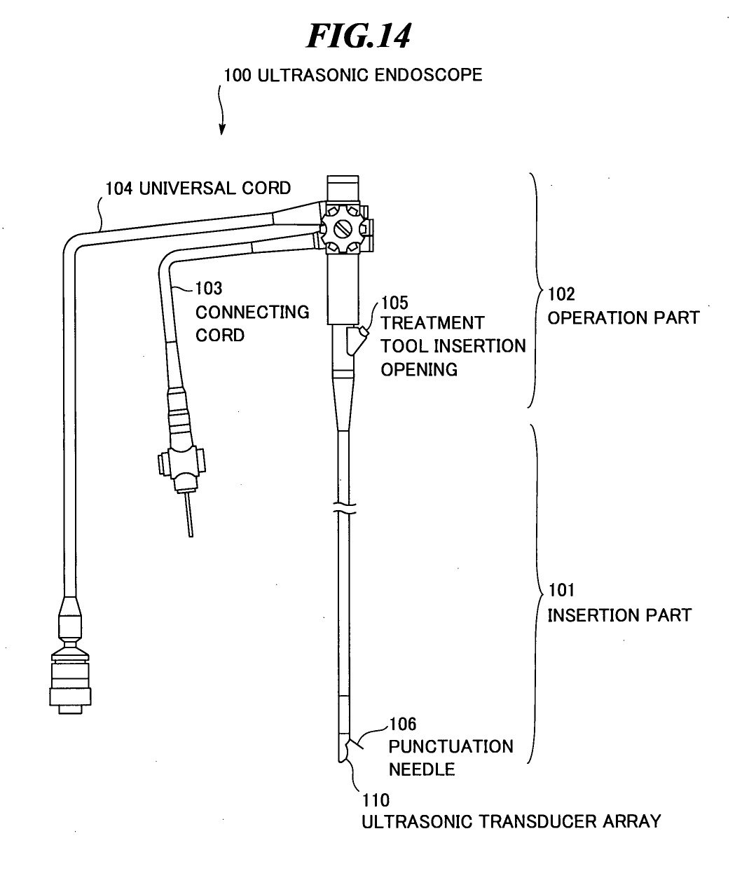

Next, an ultrasonic endoscope according to one embodiment of the present invention will be explained with reference to FIGS. 14 and 15. The ultrasonic transducer array according to the above-mentioned first to fifth embodiments is applicable not only to an ultrasonic probe to be used in contact with the object (see FIG. 2) but also an endoscope to be inserted into the object.

FIG. 14 is a schematic diagram showing an appearance of the ultrasonic endoscope. As shown in FIG. 14, the ultrasonic endoscope 100 includes an insertion part 101, an operation part 102, a connecting cord 103, and a universal cord 104.

The insertion part 101 of the ultrasonic endoscope 100 is an elongated tube formed of a material having flexibility for insertion into the body of the patient. The operation part 102 is provided at the base end of the insertion part 101, connected to the ultrasonic diagnostic apparatus main body (see FIG. 3A) via the connecting cord 103, and connected to a light source unit via the universal cord 104.

FIG. 15 is an enlarged schematic diagram showing the leading end of the insertion part 101 shown in FIG. 14. FIG. 15(a) is a side view showing a leading end of the insertion part 101, and FIG. 15(b) is a plan view showing the leading end of the insertion part 101.

As shown in FIG. 15, at the leading end of the insertion part 101, an ultrasonic transducer array 110, an observation window 111, an illumination window 112, a treatment tool passage opening 113, and a nozzle hole 114 are provided. Further, a punctuation needle 106 is provided in the treatment tool passage opening 113.

The ultrasonic transducer array 110 is a convex-type multirow array and includes five rows of elements arranged on a curved surface. Any arrangement method of elements (Fresnel arrangement or the like), size and number of layers of elements, and interconnection method (independent interconnection or common interconnection) for the ultrasonic transducer array explained in the first to fifth embodiments may be applied.

Further, as shown in FIG. 15(b), it is desirable that the ultrasonic transducer array 110 is provided such that the elevation direction is perpendicular to the insertion direction of the treatment tool (e.g., the punctuation needle 106) provided in the treatment tool passage opening 113 as seen from above. Thereby, the position of the leading end of the treatment tool in the elevation direction can be detected.

Furthermore, an acoustic matching layer is provided on the ultrasonic transmission face of the ultrasonic transducer array 110, and a backing layer is provided on the opposite face to the ultrasonic transmission face of the ultrasonic transducer array 110. In addition, an acoustic lens may be provided on the upper layer of the acoustic matching layer according to need.

An objective lens is fit in the observation window 111, and an input end of an image guide or a solid-state image sensor such as a CCD camera is provided in the imaging position of the objective lens. These configure an observation optical system. Further, an illumination lens for outputting illumination light to be supplied from the light source unit via a light guide is fit in the illumination window 112. These configure an illumination optical system.

The treatment tool passage opening 113 is a hole for leading out a treatment tool inserted from a treatment tool insertion opening 105 (FIG. 14) provided in the operation part 102. Various treatments are performed within a living cavity of the object by projecting the treatment tool such as the punctuation needle 106 or forceps from the hole and operating it in the operation part 102. Furthermore, the nozzle hole 114 is provided for injecting a liquid (water or the like) for cleaning the observation window 111 and the illumination window 112.

In the case where one of the ultrasonic transducer arrays explained in the first to fifth embodiments of the present invention is applied to the ultrasonic endoscope, three-dimensional images on a region of interest with good image quality can be acquired in real time. Referring to such ultrasonic images, a practitioner (a doctor or the like) can accurately grasp the relative position between the treatment tool (e.g., the punctuation needle 106) and an affected part. Thereby, even when the punctuation needle 106 is bent or the insertion direction is deflected from the proper direction, for example, the practitioner can perform treatment accurately and easily.

Here, in FIG. 15, the convex-type multirow array is shown as the ultrasonic transducer array 110, however, a cylindrical (radial-type) multirow array formed by further curving the multirow array and a spherical array curved not only in the azimuth direction but also in the elevation direction may be applied to the ultrasonic endoscope.

In the above-explained embodiments of the present invention, the Fresnel arrangement is used as the arrangements of elements in the elevation direction of the multirow array, however, other arrangements such as MIAE (Minimum Integrated Absolute time-delay Error) arrangement may be used. Further, the number of rows (the number of elements arranged in the elevation direction), the number of columns (the number of elements arranged in the azimuth direction), and the width and length of the entire ultrasonic transducer array may be arbitrarily designed. That is, the present invention is applicable to the case where plural kinds of elements having different areas of ultrasonic transmission/reception surfaces are arranged in one ultrasonic transducer.

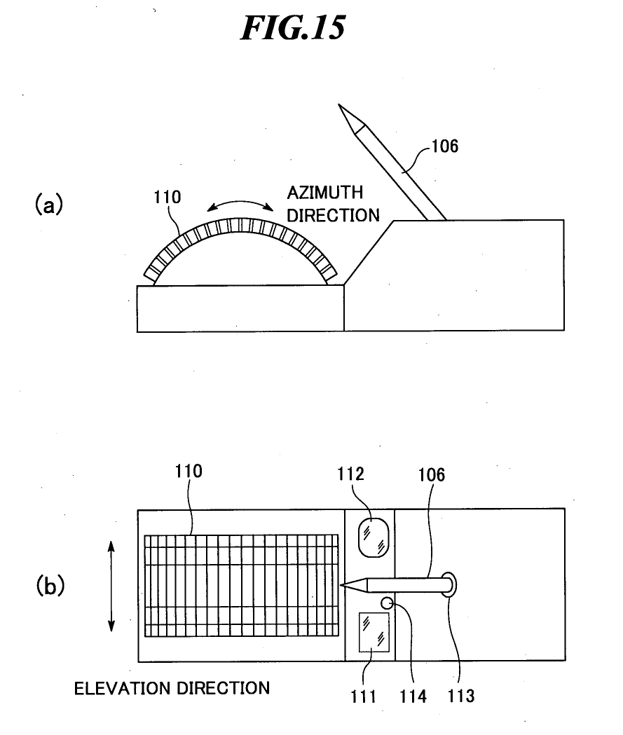

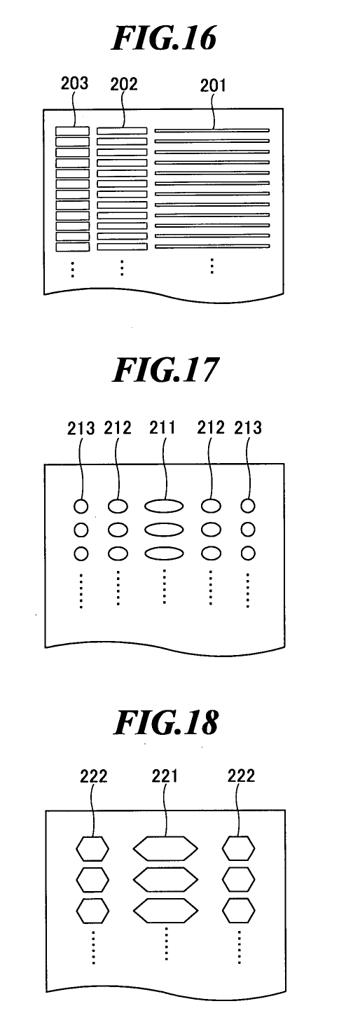

Specifically, the present invention may be applied not only to the multirow array in which the element arrangement is symmetric with respect to the center of the elevation direction is explained but also to an ultrasonic transducer array in which plural kinds of elements 201-203 are asymmetrically arranged as shown in FIG. 16.

Further, the number of rows in which the elements are arranged is not limited to five, but the present invention may be applied to a multirow array having at least two rows. Furthermore, in the above-mentioned embodiments, the arrangement pitch in the elevation direction is equal to or more than the wavelength of transmission ultrasonic waves, however, it may be less than the wavelength of transmission ultrasonic waves.

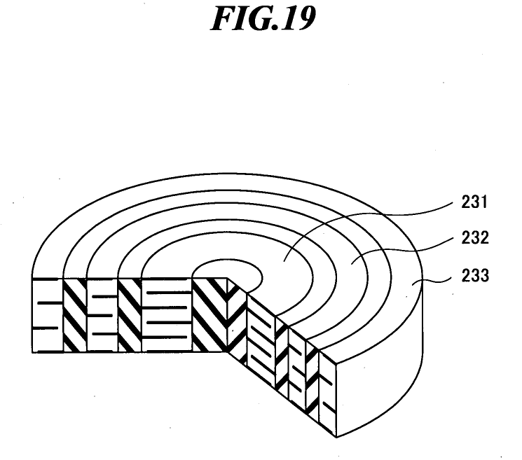

On the other hand, regarding the shape of elements, not only the rectangular shape but also an arbitrary shape may be used. As shown in FIG. 17, oval elements 211-213 may be arranged in multi row. Or, as shown in FIG. 18, polygonal (e.g., hexagonal) elements 221 and 222 may be arranged in multirow. Furthermore, as shown in FIG. 19, annular elements 231-233 may be arranged in a concentric pattern or plural elements may be annularly arranged. Alternatively, the shapes of plural kinds of elements arranged in one ultrasonic transducer array may be different from one another. In any case, the number of layers of elements are designed such that electric impedances are substantially made equal among the plural kinds of elements having different areas of ultrasonic transmission/reception surfaces, and therefore, variation in transmission acoustic energy and variation in reception sensitivity can be reduced.

Further, in the embodiment, at a maximum two elements are commonly interconnected, however, three or more elements may be commonly interconnected. In this case, the number of layers of the respective elements may be determined according to the total area of ultrasonic transmission/reception surfaces such that the synthesized electric impedances with respect to each interconnection become substantially the same. Furthermore, since the Fresnel arrangement is adopted in FIGS. 8 and 10, the area of the ultrasonic transmission/reception surface of the independently interconnected element (e.g., element 31) is larger than the total area of the ultrasonic transmission/reception surfaces of the commonly interconnected two elements (e.g., elements 32). However, the arrangement method is not limited to the Fresnel arrangement, and therefore, the area of the ultrasonic transmission/reception surface of the independently interconnected element can be sometimes larger than the total area of the ultrasonic transmission/reception surfaces of the commonly interconnected plural elements. In such a case, the synthesized electric impedances with respect to interconnection can be made substantially the same by increasing the number of layers of the elements having the smaller areas ultrasonic transmission/reception surfaces or decreasing the number of layers of the groups of elements having the larger total area of ultrasonic transmission/reception surfaces.

In addition, the arrangement surface of elements in the ultrasonic transducer array may be a flat surface as shown in FIG. 1, a convex surface as shown in FIG. 15, a concave surface, a spherical surface, or any other curved surfaces.

1. An ultrasonic transducer array in which at least two kinds of ultrasonic transducers having different areas of ultrasonic transmission/reception surfaces from one another and having substantially equal total thicknesses to one another are arranged, said ultrasonic transducer array comprising:
a first ultrasonic transducer; and
a second ultrasonic transducer having a larger number of layers than that of said first ultrasonic transducer and a smaller area of an ultrasonic transmission/reception surface than that of said first ultrasonic transducer.

2. The ultrasonic transducer array according to claim 1, wherein an electric impedance of said first ultrasonic transducer and an electric impedance of said second ultrasonic transducer are substantially equal to each other.

3. The ultrasonic transducer array according to claim 2, wherein S₁:S₂=1/N₁²:1/N₂², where N₁ represents the number of layers of said first ultrasonic transducer, S₁ represents the area of the ultrasonic transmission/reception surface thereof, N₂ represents the number of layers of said second ultrasonic transducer, and S₂ represents the area of the ultrasonic transmission/reception surface thereof.

4. An ultrasonic transducer array in which at least two kinds of ultrasonic transducers having substantially equal total thicknesses to one another are arranged, said ultrasonic transducer array comprising:
at least one first ultrasonic transducer connected to a first interconnection; and
at least one second ultrasonic transducer connected to a second interconnection, having a larger number of layers than that of said at least one first ultrasonic transducer and a smaller total area of ultrasonic transmission/reception surface than that of said first ultrasonic transducer.

5. The ultrasonic transducer array according to claim 4, wherein a total capacitance of said at least one first ultrasonic transducer and a total capacitance of said at least one second ultrasonic transducer are substantially equal to each other.

6. The ultrasonic transducer array according to claim 4, wherein S₁:S₂=1/(N₁²·M₁):1/(N₂²·M₂), where N₁ represents the number of layers of said at least one first ultrasonic transducer, S₁ represents the total area of the ultrasonic transmission/reception surface thereof, M₁ represents the number of said at least one first ultrasonic transducer connected to the first interconnection, N₂ represents the number of layers of said at least one second ultrasonic transducer, S₂ represents the total area of the ultrasonic transmission/reception surface thereof, and M₂ represents the number of said at least one second ultrasonic transducer connected to the second interconnection.

7. The ultrasonic transducer array according to claim 1, comprising:
plural first ultrasonic transducers one-dimensionally arranged in a first element row; and
plural second ultrasonic transducers one-dimensionally arranged in a second element row in parallel with the first element row.

8. The ultrasonic transducer array according to claim 4 comprising:
plural first ultrasonic transducers one-dimensionally arranged in a first element row; and
plural second ultrasonic transducers one-dimensionally arranged in a second element row in parallel with the first element row.

9. The ultrasonic transducer array according to claim 7, wherein each of said plural first and second ultrasonic transducers has a rectangular ultrasonic transmission/reception surface.

10. The ultrasonic transducer array according to claim 7, wherein a width of an ultrasonic transducer arranged in an outer element row is narrower than that of an ultrasonic transducer arranged in an inner element row in a direction perpendicular to an arrangement direction of ultrasonic transducers in each element row.

11. The ultrasonic transducer array according to claim 7, wherein an arrangement pitch of said plural first ultrasonic transducers in said first element row and an arrangement pitch of said plural second ultrasonic transducers in said second element row are equal to each other.

12. The ultrasonic transducer array according to claim 7, wherein an interval between said plural first ultrasonic transducers in said first element row and an interval between said plural second ultrasonic transducers in said second element row are equal to each other.

13. The ultrasonic transducer array according to claim 7, wherein an arrangement pitch of ultrasonic transducers arranged in an inner element row is less than that of ultrasonic transducers arranged in an outer element row.

14. An ultrasonic probe comprising:
an ultrasonic transducer array in which at least two kinds of ultrasonic transducers having different areas of ultrasonic transmission/reception surfaces from one another and having substantially equal total thicknesses to one another are arranged, said ultrasonic transducer array including a first ultrasonic transducer, and a second ultrasonic transducer having a larger number of layers than that of said first ultrasonic transducer and a smaller area of an ultrasonic transmission/reception surface than that of said first ultrasonic transducer;
an acoustic matching layer provided at an ultrasonic transmission/reception surface side of said ultrasonic transducer array; and
a backing layer provided at an opposite side to said ultrasonic transmission/reception surface side of said ultrasonic transducer array.

15. An ultrasonic probe comprising:
an ultrasonic transducer array in which at least two kinds of ultrasonic transducers having substantially equal total thicknesses to one another are arranged, said ultrasonic transducer array including at least one first ultrasonic transducer connected to a first interconnection, and at least one second ultrasonic transducer connected to a second interconnection, having a larger number of layers than that of said at least one first ultrasonic transducer and a smaller total area of ultrasonic transmission/reception surface than that of said first ultrasonic transducer;
an acoustic matching layer provided at an ultrasonic transmission/reception surface side of said ultrasonic transducer array; and
a backing layer provided at an opposite side to said ultrasonic transmission/reception surface side of said ultrasonic transducer array.

16. The ultrasonic probe according to claim 14, further comprising:
correcting means for correcting a level difference between a reception signal outputted from said first ultrasonic transducer and a reception signal outputted from said second ultrasonic transducer.

17. The ultrasonic probe according to claim 15, further comprising:
correcting means for correcting a level difference between a reception signal outputted from said first ultrasonic transducer and a reception signal outputted from said second ultrasonic transducer.

18. An ultrasonic endoscope to be used by being inserted into a body of a patient, said ultrasonic endoscope comprising:
an insertion part formed of a material having flexibility and to be inserted into the body of the patient; and
an ultrasonic transducer array provided at a leading end of said insertion part in which at least two kinds of ultrasonic transducers having different areas of ultrasonic transmission/reception surfaces from one another and having substantially equal total thicknesses to one another are arranged, said ultrasonic transducer array including a first ultrasonic transducer, and a second ultrasonic transducer having a larger number of layers than that of said first ultrasonic transducer and a smaller area of an ultrasonic transmission/reception surface than that of said first ultrasonic transducer.

19. An ultrasonic endoscope to be used by being inserted into a body of a patient, said ultrasonic endoscope comprising:
an insertion part formed of a material having flexibility and to be inserted into the body of the patient; and
an ultrasonic transducer array provided at a leading end of said insertion part in which at least two kinds of ultrasonic transducers having substantially equal total thicknesses to one another are arranged, said ultrasonic transducer array including at least one first ultrasonic transducer connected to a first interconnection, and at least one second ultrasonic transducer connected to a second interconnection, having a larger number of layers than that of said at least one first ultrasonic transducer and a smaller total area of ultrasonic transmission/reception surface than that of said first ultrasonic transducer.

20. The ultrasonic endoscope according to claim 18, further comprising:
a treatment tool to be passed through said insertion part and inserted from an opening provided at the leading end of said insertion part into the body of the patient;
wherein said ultrasonic transducer array includes plural first ultrasonic transducers one-dimensionally arranged in a first element row, and plural second ultrasonic transducers one-dimensionally arranged in a second element row in parallel with the first element row, and plural ultrasonic transducers in each element row are arranged to be able to detect a position of a leading end of said treatment tool.

21. The ultrasonic endoscope according to claim 19, further comprising:
a treatment tool to be passed through said insertion part and inserted from an opening provided at the leading end of said insertion part into the body of the patient;
wherein said ultrasonic transducer array includes plural first ultrasonic transducers one-dimensionally arranged in a first element row, and plural second ultrasonic transducers one-dimensionally arranged in a second element row in parallel with the first element row, and plural ultrasonic transducers in each element row are arranged to be able to detect a position of a leading end of said treatment tool.

22. The ultrasonic endoscope according to claim 18, further comprising:
correcting means for correcting a level difference between a reception signal outputted from said first ultrasonic transducer and a reception signal outputted from said second ultrasonic transducer.

23. The ultrasonic endoscope according to claim 19, further comprising:
correcting means for correcting a level difference between a reception signal outputted from said first ultrasonic transducer and a reception signal outputted from said second ultrasonic transducer.

24. An ultrasonic diagnostic apparatus comprising:
an ultrasonic probe including (i) an ultrasonic transducer array in which at least two kinds of ultrasonic transducers having different areas of ultrasonic transmission/reception surfaces from one another and having substantially equal total thicknesses to one another are arranged, said ultrasonic transducer array including a first ultrasonic transducer, and a second ultrasonic transducer having a larger number of layers than that of said first ultrasonic transducer and a smaller area of an ultrasonic transmission/reception surface than that of said first ultrasonic transducer, (ii) an acoustic matching layer provided at an ultrasonic transmission/reception surface side of said ultrasonic transducer array, and (iii) a backing layer provided at an opposite side to said ultrasonic transmission/reception surface side of said ultrasonic transducer array;
a drive signal generating unit for generating drive signals to be respectively supplied to said first and second ultrasonic transducers;
a signal processing unit for processing reception signals respectively outputted from said plural first and second ultrasonic transducers; and
an image generating unit for generating an ultrasonic image based on the reception signals processed by said signal processing unit.

25. An ultrasonic diagnostic apparatus comprising:
an ultrasonic probe including (i) an ultrasonic transducer array in which at least two kinds of ultrasonic transducers having substantially equal total thicknesses to one another are arranged, said ultrasonic transducer array including at least one first ultrasonic transducer connected to a first interconnection, and at least one second ultrasonic transducer connected to a second interconnection, having a larger number of layers than that of said at least one first ultrasonic transducer and a smaller total area of ultrasonic transmission/reception surface than that of said first ultrasonic transducer, (ii) an acoustic matching layer provided at an ultrasonic transmission/reception surface side of said ultrasonic transducer array, and (iii) a backing layer provided at an opposite side to said ultrasonic transmission/reception surface side of said ultrasonic transducer array;
a drive signal generating unit for generating drive signals to be respectively supplied to said first and second ultrasonic transducers;
a signal processing unit for processing reception signals respectively outputted from said plural first and second ultrasonic transducers; and
an image generating unit for generating an ultrasonic image based on the reception signals processed by said signal processing unit.

26. An ultrasonic diagnostic apparatus comprising:
an ultrasonic endoscope to be used by being inserted into a body of a patient, said ultrasonic endoscope including (i) an insertion part formed of a material having flexibility and to be inserted into the body of the patient, and (ii) an ultrasonic transducer array provided at a leading end of said insertion part in which at least two kinds of ultrasonic transducers having different areas of ultrasonic transmission/reception surfaces from one another and having substantially equal total thicknesses to one another are arranged, said ultrasonic transducer array including a first ultrasonic transducer, and a second ultrasonic transducer having a larger number of layers than that of said first ultrasonic transducer and a smaller area of an ultrasonic transmission/reception surface than that of said first ultrasonic transducer;
a drive signal generating unit for generating drive signals to be respectively supplied to said first and second ultrasonic transducers;
a signal processing unit for processing reception signals respectively outputted from said plural first and second ultrasonic transducers; and
an image generating unit for generating an ultrasonic image based on the reception signals processed by said signal processing unit.

27. An ultrasonic diagnostic apparatus comprising:
an ultrasonic endoscope to be used by being inserted into a body of a patient, said ultrasonic endoscope including (i) an insertion part formed of a material having flexibility and to be inserted into the body of the patient, and (ii) an ultrasonic transducer array provided at a leading end of said insertion part in which at least two kinds of ultrasonic transducers having substantially equal total thicknesses to one another are arranged, said ultrasonic transducer array including at least one first ultrasonic transducer connected to a first interconnection, and at least one second ultrasonic transducer connected to a second interconnection, having a larger number of layers than that of said at least one first ultrasonic transducer and a smaller total area of ultrasonic transmission/reception surface than that of said first ultrasonic transducer;
a drive signal generating unit for generating drive signals to be respectively supplied to said first and second ultrasonic transducers;
a signal processing unit for processing reception signals respectively outputted from said plural first and second ultrasonic transducers; and
an image generating unit for generating an ultrasonic image based on the reception signals processed by said signal processing unit.

28. The ultrasonic diagnostic apparatus according to claim 24, wherein said signal processing unit includes:
correcting means for correcting a level difference between a reception signal outputted from said first ultrasonic transducer and a reception signal outputted from said second ultrasonic transducer.

29. The ultrasonic diagnostic apparatus according to claim 25, wherein said signal processing unit includes:
correcting means for correcting a level difference between a reception signal outputted from said first ultrasonic transducer and a reception signal outputted from said second ultrasonic transducer.

30. The ultrasonic diagnostic apparatus according to claim 26, wherein said signal processing unit includes:
correcting means for correcting a level difference between a reception signal outputted from said first ultrasonic transducer and a reception signal outputted from said second ultrasonic transducer.

31. The ultrasonic diagnostic apparatus according to claim 27, wherein said signal processing unit includes:
correcting means for correcting a level difference between a reception signal outputted from said first ultrasonic transducer and a reception signal outputted from said second ultrasonic transducer.

32. The ultrasonic diagnostic apparatus according to claim 28, wherein said correcting means includes plural preamplifiers for pre-amplifying reception signals respectively outputted from said first and second ultrasonic transducers, said plural preamplifiers having gains respectively set according to the numbers of layers and/or the areas of ultrasonic transmission/reception surfaces of said first and second ultrasonic transducers.

33. The ultrasonic diagnostic apparatus according to claim 29, wherein said correcting means includes plural preamplifiers for pre-amplifying reception signals respectively outputted from said first and second ultrasonic transducers, said plural preamplifiers having gains respectively set according to the numbers of layers and/or the areas of ultrasonic transmission/reception surfaces of said first and second ultrasonic transducers.

34. The ultrasonic diagnostic apparatus according to claim 30, wherein said correcting means includes plural preamplifiers for pre-amplifying reception signals respectively outputted from said first and second ultrasonic transducers, said plural preamplifiers having gains respectively set according to the numbers of layers and/or the areas of ultrasonic transmission/reception surfaces of said first and second ultrasonic transducers.

35. The ultrasonic diagnostic apparatus according to claim 31, wherein said correcting means includes plural preamplifiers for pre-amplifying reception signals respectively outputted from said first and second ultrasonic transducers, said plural preamplifiers having gains respectively set according to the numbers of layers and/or the areas of ultrasonic transmission/reception surfaces of said first and second ultrasonic transducers.

36. The ultrasonic diagnostic apparatus according to claim 28, wherein said correcting means includes plural variable gain circuits for adjusting levels of reception signals outputted from said first and second ultrasonic transducers and pre-amplified, said plural variable gain circuits having gains respectively set according to the numbers of layers and/or the areas of ultrasonic transmission/reception surfaces of said first and second ultrasonic transducers.

37. The ultrasonic diagnostic apparatus according to claim 29, wherein said correcting means includes plural variable gain circuits for adjusting levels of reception signals outputted from said first and second ultrasonic transducers and pre-amplified, said plural variable gain circuits having gains respectively set according to the numbers of layers and/or the areas of ultrasonic transmission/reception surfaces of said first and second ultrasonic transducers.

38. The ultrasonic diagnostic apparatus according to claim 30, wherein said correcting means includes plural variable gain circuits for adjusting levels of reception signals outputted from said first and second ultrasonic transducers and pre-amplified, said plural variable gain circuits having gains respectively set according to the numbers of layers and/or the areas of ultrasonic transmission/reception surfaces of said first and second ultrasonic transducers.

39. The ultrasonic diagnostic apparatus according to claim 31, wherein said correcting means includes plural variable gain circuits for adjusting levels of reception signals outputted from said first and second ultrasonic transducers and pre-amplified, said plural variable gain circuits having gains respectively set according to the numbers of layers and/or the areas of ultrasonic transmission/reception surfaces of said first and second ultrasonic transducers.

40. The ultrasonic diagnostic apparatus according to claim 28, wherein said correcting means includes processing means for performing arithmetic processing on reception signals outputted from said first and second ultrasonic transducers and A/D converted, said processing means correcting levels of the respective reception signals with correction factors according to the numbers of layers and/or the areas of ultrasonic transmission/reception surfaces of said first and second ultrasonic transducers.

41. The ultrasonic diagnostic apparatus according to claim 29, wherein said correcting means includes processing means for performing arithmetic processing on reception signals outputted from said first and second ultrasonic transducers and A/D converted, said processing means correcting levels of the respective reception signals with correction factors according to the numbers of layers and/or the areas of ultrasonic transmission/reception surfaces of said first and second ultrasonic transducers.

42. The ultrasonic diagnostic apparatus according to claim 30, wherein said correcting means includes processing means for performing arithmetic processing on reception signals outputted from said first and second ultrasonic transducers and A/D converted, said processing means correcting levels of the respective reception signals with correction factors according to the numbers of layers and/or the areas of ultrasonic transmission/reception surfaces of said first and second ultrasonic transducers.

43. The ultrasonic diagnostic apparatus according to claim 31, wherein said correcting means includes processing means for performing arithmetic processing on reception signals outputted from said first and second ultrasonic transducers and A/D converted, said processing means correcting levels of the respective reception signals with correction factors according to the numbers of layers and/or the areas of ultrasonic transmission/reception surfaces of said first and second ultrasonic transducers.

44. The ultrasonic diagnostic apparatus according to claim 28, wherein said correcting means corrects the reception signal outputted from said first ultrasonic transducer and the reception signal outputted from said second ultrasonic transducer such that a ratio of gains or correction factors is N₁:N₂, where N₁ represents the number of layers of said first ultrasonic transducer and N₂ represents the number of layers of said second ultrasonic transducer.

45. The ultrasonic diagnostic apparatus according to claim 29, wherein said correcting means corrects the reception signal outputted from said first ultrasonic transducer and the reception signal outputted from said second ultrasonic transducer such that a ratio of gains or correction factors is N₁:N₂, where N₁ represents the number of layers of said first ultrasonic transducer and N₂ represents the number of layers of said second ultrasonic transducer.

46. The ultrasonic diagnostic apparatus according to claim 30, wherein said correcting means corrects the reception signal outputted from said first ultrasonic transducer and the reception signal outputted from said second ultrasonic transducer such that a ratio of gains or correction factors is N₁:N₂, where N₁ represents the number of layers of said first ultrasonic transducer and N₂ represents the number of layers of said second ultrasonic transducer.

47. The ultrasonic diagnostic apparatus according to claim 31, wherein said correcting means corrects the reception signal outputted from said first ultrasonic transducer and the reception signal outputted from said second ultrasonic transducer such that a ratio of gains or correction factors is N₁:N₂, where N₁ represents the number of layers of said first ultrasonic transducer and N₂ represents the number of layers of said second ultrasonic transducer.
